(19) United States
(12) Patent Application Publication
(10) Pub. No.: US 2023/0248672 A1
(43) Pub. Date: Aug. 10, 2023

(54) COMPOSITIONS COMPRISING GARDOS CHANNEL ANTAGONISTS AND THEIR USES

(71) Applicant: ADIMABIO LLC, Wellesley, MA (US)

(72) Inventor: Santhosh VADIVELU, Boston, MA (US)

(21) Appl. No.: 18/015,592

(22) PCT Filed: Jul. 14, 2021
(86) PCT No.: PCT/US2021/041695

§ 371 Date: Jan. 11, 2023

Related U.S. Application Data
(60) Provisional application No. 63/051,770, filed on Jul. 14, 2020.

(51) Int. Cl. A61K 31/165; A61K 45/06; A61K 47/14; A61K 9/107

(52) U.S. Cl. CPC A61K 31/165; A61K 45/06; A61K 47/14; A61K 9/1075

(57) ABSTRACT

In one aspect, the disclosure herein relates to pharmaceutical compositions formulated as an oral dosage form comprising a therapeutically effective amount of at least one substituted triphenylacetamide analog, e.g., 2,2-bis(4-fluorophenyl)-2-phenylacetamide, optionally a second therapeutic agent, and methods of making same. In another aspect, the disclosure relates to methods of treating hematological disorders, e.g., a disorder associated with a sickling blood disorder and/or a thalassemia, by administering the disclosed pharmaceutical compositions to a subject. In a still further aspect, the disclosure relates to kits comprising at least one substituted triphenylacetamide analog, e.g., 2,2-bis(4-fluorophenyl)-2-phenylacetamide, useful for treating hematological disorders and diseases associated with a sickling blood disorder and/or a thalassemia. This abstract is intended as a scanning tool for purposes of searching in the particular art and is not intended to be limiting of the present disclosure.

### CROSS-REFERENCE TO RELATED APPLICATIONS

This application claims the benefit of U.S. Provisional Application No. 63/051,770, filed on Jul. 14, 2020, which is incorporated herein by reference in its entirety.

### BACKGROUND

Sickle cell disease is an inherited condition in which mutated hemoglobin polymerizes in red blood cells. This polymerization causes red blood cells to adopt a sickle or crescent shape which can lead to premature death of red blood cells and thus insufficient, numbers of healthy red blood cells, in turn causing pain, increased susceptibility to infection, fatigue, and blockage of blood vessels. Individuals with sickle cell disease are at increased risk of stroke and clotting disorders, blindness, gallstones: pulmonary hypertension, pregnancy complications, and organ damage and many of those affected die prematurely.

The major source of morbidity and mortality of patients suffering from sickle cell disease is vascular occlusion caused by the sickled cells, which causes repeated episodes of pain in both acute and chronic form and also causes ongoing organ damage with the passage of time. It has long been recognized and accepted that the deformation and distortion of sickle cell erythrocytes upon complete deoxygenation is caused by polymerization and intracellular gelation of sickle hemoglobin, hemoglobin S (HbS), The phenomenon is well reviewed and discussed by Eaton et al., Blood 70:1245 (1987). The intracellular gelatin and polymerization of Hb S can occur at any time during an erythrocyte's journey through the vasculature. Thus, erythrocytes in patients with sickle cell disease containing no polymerized hemoglobin S may pass through the microcirculation and return to the lungs without sickling, may sickle in the veins or may sickle in the capillaries.

Many approaches to therapeutically treating dehydrated sickle cells (thus decreasing polymerization of hemoglobin S by lowering the osmolality of plasma) have been tried with limited success, including the following approaches: intravenous infusion of distilled water (Gye et al., Am. J Med. Sci. 266: 267-277(1973)); administration of the antidiuretic hormone vasopressin together with a high fluid intake and salt restriction (Rosa et at, M Eng. J Med. 303:1138-1143 (1980)); Charache et al., Blood 58: 892-896 (1981)); the use of monensin to increase the cation content of the sickle cell (Clark et al., J. Clin. Invest. 70:1074-1080 (1982)); Fahim et al., Life Sciences 29:1959-1966 (1981)); intravenous administration of cetiedil citrate (Benjamin et al., Blood 67: 1442-1447 (1986)); Berkowitz et al., Am. J. Hematol. 17: 217-223 (1984)); Stuart et al., J Clin. Pathol. 40:1182-1186 (1987)); and the use of oxpentifylline (Stuart et al., supra).

Another approach towards therapeutically treating dehydrated sickle cells involves altering erythrocyte potassium flux by targeting a calcium-dependent potassium channel (Ishi et al., Proc. Natl. Acad. Sci. 94(21): 11651-6 (1997)), currently available therapeutic methods targeted to the Gardos channel suffer from clinical shortcomings such as poor bioavailability and unacceptable toxicities.

Despite advances in research directed to treatment of sickle cell disease and related hematological disorders, there is still a scarcity of pharmaceutical compositions that are bioavailable, potent, efficacious, and selective antagonists of the Gardos channel. These needs and other needs are satisfied by the present disclosure.

### SUMMARY

In accordance with the purpose(s) of the disclosure, as embodied and broadly described herein, the disclosure, in one aspect, relates to pharmaceutical compositions formulated as an oral dosage form comprising a therapeutically effective amount of at least one substituted triphenylacetamide analog, e.g., 2,2-bis(4-fluorophenyl)-2-phenylacetamide, optionally a second therapeutic agent, and methods of making same. In another aspect, the disclosure relates to methods of treating a disorder, such as a hematological disorder, a neurological disorder, a viral infection, or a cancer, by administering the disclosed pharmaceutical compositions to a subject. In a still further aspect, the disclosure relates to kits comprising at least one substituted triphenylacetamide analog, e.g., 2,2-bis(4-fluorophenyl)-2-phenylacetamide, useful for treating disorder, such as a hematological disorder, a neurological disorder, a viral infection, or a cancer. In a further aspect, the disclosed pharmaceutical compositions comprise a second therapeutic agent as disclosed herein. Examples of hematological disorders, as used herein, include a sickling blood disorder, a thalassemia, or an anemia, e.g., hereditary stomacytosis.

Disclosed are pharmaceutical compositions comprising a therapeutically effective amount of at least one substituted triphenyl acetamide analog having a structure represented by a formula:

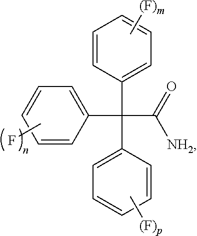

wherein m, n and p can be independently selected from 0 and 1 and at least one of m, n and p can be 1, or a pharmaceutically acceptable salt, solvate, or polymorph thereof; optionally one or more additional therapeutic agents; and a pharmaceutically acceptable carrier. In a further aspect, the least one substituted triphenyl acetamide analog has a structure represented by a formula:

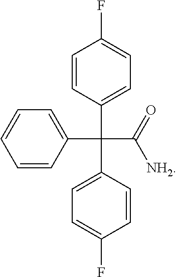 [SMILES: NC(=O)C(c1ccccc1)(c1ccc(F)cc1)c1ccc(F)cc1]

Disclosed are pharmaceutical compositions comprising a therapeutically effective amount of at least one substituted triphenyl acetamide analog having a structure represented by a formula:

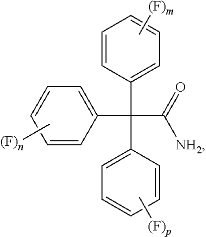

wherein m, n and p can be independently selected from 0 and 1 and at least one of m, n and p can be 1, or a pharmaceutically acceptable salt, solvate, or polymorph thereof; optionally one or more additional therapeutic agents; and a pharmaceutically acceptable carrier; wherein the pharmaceutically acceptable carrier comprises at least one oil or lipidic material, at least one surfactant, and at least one hydrophilic co-surfactant; and wherein the least one oil or lipidic material, the least one surfactant, and the least one hydrophilic co-surfactant form a suspension, an emulsion, or a microemulsion. In a further aspect, the foregoing pharmaceutical composition is a microemulsion. In a still further aspect, the microemulsion is a self-emulsifying microemulsion.

Disclosed are pharmaceutical compositions comprising a therapeutically effective amount of at least one substituted triphenyl acetamide analog having a structure represented by a formula:

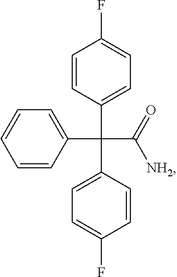 [SMILES: NC(=O)C(c1ccccc1)(c1ccc(F)cc1)c1ccc(F)cc1]

optionally one or more additional therapeutic agents; and a pharmaceutically acceptable carrier; wherein the pharmaceutically acceptable carrier comprises at least one oil or lipidic material, at least one surfactant, and at least one hydrophilic co-surfactant; and wherein the least one oil or lipidic material, the least one surfactant, and the least one hydrophilic co-surfactant form a suspension, an emulsion, or a microemulsion. In a further aspect, the foregoing pharmaceutical composition is a microemulsion. In a still further aspect, the microemulsion is a self-emulsifying microemulsion.

Also disclosed are pharmaceutical compositions formulated as a dosage form comprising a disclosed suspension, emulsion, or microemulsion within an enteric coating formulated as a capsule.

Also disclosed are methods for the treatment of a disorder, such as a hematological disorder, a neurological disorder, a viral infection, or a cancer, in a mammal comprising the step of administering to the mammal a therapeutically effective amount of at least one disclosed pharmaceutical composition.

Also disclosed are methods for the treatment of a disorder, such as a hematological disorder, a neurological disorder, a viral infection, or a cancer, in a mammal comprising the step of administering to the mammal co-administration of a therapeutically effective amount of at least one disclosed pharmaceutical composition comprising at least one substituted triphenylacetamide analog, e.g., 2,2-bis(4-fluorophenyl)-2-phenylacetamide, and a disclosed second therapeutic agent.

Also disclosed are methods for the treatment of a hematological disorder, e.g., a sickling blood disorder, a thalassemia, or an anemia, e.g., hereditary stomacytosism, in a mammal comprising the step of administering to the mammal a therapeutically effective amount of at least one disclosed pharmaceutical composition.

Also disclosed are methods for the treatment of a hematological disorder, e.g., a sickling blood disorder, a thalassemia, or an anemia, e.g., hereditary stomacytosis, in a mammal comprising the step of administering to the mammal co-administration of a therapeutically effective amount of at least one disclosed pharmaceutical composition comprising at least one substituted triphenylacetamide analog, e.g., 2,2-bis(4-fluorophenyl)-2-phenylacetamide, and a disclosed second therapeutic agent.

Also disclosed are uses of a disclosed pharmaceutical composition.

Also disclosed are uses of a disclosed pharmaceutical composition in the manufacture of a medicament for the treatment of a hematological disorders and diseases associated with a sickling blood disorder and/or a thalassemia in a mammal.

Also disclosed are kits comprising at least one substituted triphenylacetamide analog. e.g., 2,2-bis(4-fluorophenyl)-2-phenylacetamide, and one or more of (a) at least one additional therapeutic agent; or (b) instructions for treating a hematological disorder or disease associated with a sickling blood disorder and/or a thalassemia in a mammal.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

Additional advantages of the disclosure will be set forth in part in the description which follows, and in part will be obvious from the description, or can be learned by practice of the disclosure. The advantages of the disclosure will be realized and attained by means of the elements and combinations particularly pointed out in the appended claims. It is to be understood that both the foregoing general description and the following detailed description are exemplary and explanatory only and are not restrictive of the disclosure, as claimed.

### DETAILED DESCRIPTION

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present disclosure is not entitled to antedate such publication by virtue of prior disclosure. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a second therapeutic agent,” “a triphenyl acetamide analog,” or “a pharmaceutical carrier,” includes, but is not limited to, combinations of two or more such second therapeutic agents, triphenyl acetamide analogs, or pharmaceutical carriers, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

Where a range is expressed, a further aspect includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about ‘about y’, and ‘about z’ as well as the ranges of ‘less than x’, less than y’, and ‘less than z’. Likewise, the phrase ‘about x, y, z, or greater’ should be interpreted to include the specific ranges of ‘about ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y”’.

It is to be understood that Such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

Disclosed are the components to be used to prepare the compositions of the disclosure as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to the compound are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the disclosure. Thus, if there are a variety of additional steps that can be performed it is understood that each of these additional steps can be performed with any specific embodiment or combination of embodiments of the methods of the disclosure.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not.

As used herein, “senicapoc”, “ICA-17043” and “2,2-bis(4-fluorophenyl)-2-phenylacetamide” can be used interchangeably, and refer to a substituted triphenylacetamide analog having a structure given by the formula:

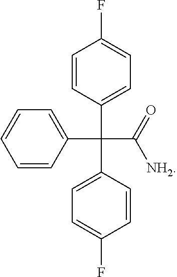 [SMILES: NC(=O)C(c1ccccc1)(c1ccc(F)cc1)c1ccc(F)cc1]

As used herein, “GBT440”, “GST-440”, “GBT 440”, “Oxbryta®”, “voxelotor”, and “2-hydroxy-6-[[2-(2-propan-2-ylpyrazol-3-yl)pyridin-3-yl]methoxy]benzaldehyde” can be used interchangeably and refer to the same therapeutic agent that targets and covalently binds to the N-terminal valine of the alpha chain of HbS. This stabilizes HbS, thereby improving oxygen binding affinity. The binding of voxelotor to HbS prevents HbS polymerization, reduces sickling, decreases red blood cell (RBC) damage and increases the half-life of RBCs. This improves blood flow and decreases hemolytic anemia.

As used herein, the term “self-emulsifying” describes a system in which emulsifies when mixed with an aqueous solvent and which, upon exposure to gastrointestinal fluids, forms stable-microemulsions.

As used herein, the term “self-microemulsifying” describes a system in which emulsifies when mixed with an aqueous solvent and which, upon exposure to gastrointestinal fluids, forms stable-microemulsions with diameters in the range of 1 μm.

As used herein, the terms “SNEDD” and “self nanoemulsifying drug delivery system” can be used interchangeably and refer to self emulsifying isotropic mixtures comprising oil, surfactant, and co-surfactant. In a SNEDD, when combined with the aqueous phase in the gastrointestinal tract, the droplet size is below 200 nm.

As used herein, “administering” can refer to an administration to a subject of one or more therapeutic agents by a route of administration that can be oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. For example a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used interchangeably herein, “subject,” “individual,” or “patient” can refer to a vertebrate organism, such as a mammal (e.g. human). “Subject” can also refer to a cell, a population of cells, a tissue, an organ, or an organism, preferably to human and constituents thereof.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as sickle cell disease, thalassemia, and/or a cancer. The effect can be therapeutic in terms of a partial or complete cure of a disease, condition, symptom or adverse effect attributed to the disease, disorder, or condition. The term “treatment” as used herein can include any treatment of sickle cell disease, thalassemia, and/or a cancer in a subject, particularly a human and can include any one or more of the following: (a) preventing the disease from occurring in a subject which may be predisposed to the disease but has not yet been diagnosed as having it; (b) inhibiting the disease, arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, such as treating the pain of a subject by administration of an analgesic agent even though such agent does not treat the cause of the pain. In one aspect, treatment of sickle cell disease or thalassemia can increase total hemoglobin or increase fetal hemoglobin or reduce anemia or reduce clumping among misshapen erythrocytes.

As used herein, the term“prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, “therapeutic” can refer to treating, healing, and/or ameliorating a disease, disorder, condition, or side effect, or to decreasing in the rate of advancement of a disease, disorder, condition, or side effect.

As used herein, “therapeutic agent” can refer to any substance, compound, molecule, and the like, which can be biologically active or otherwise can induce a pharmacologic, immunogenic, biologic and/or physiologic effect on a subject to which it is administered to by local and/or systemic action. A therapeutic agent can be a primary active agent, or in other words, the components) of a composition to which the whole or part of the effect of the composition is attributed. A therapeutic agent can be a second therapeutic agent, or in other words, the component(s) of a composition to which an additional part and/or other effect of the composition is attributed. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14th edition), the Physicians' Desk Reference (64th edition), and The Pharmacological Basis of Therapeutics (12th edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications, as well as other areas. The term therapeutic agent also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

As used herein, “therapeutically effective amount” refers to the amount of a disclosed compound, e.g., at least one substituted triphenylacetamide analog, e.g., 2,2-bis(4-fluorophenyl)-2-phenylacetamide, in its crystalline or amorphous form, as well as its esters, salts, or derivatives thereof, or pharmaceutical composition provided herein that is sufficient to effect beneficial or desired biological, emotional, medical, or clinical response of a cell, tissue, system, animal, or human. An effective amount can be administered in one or more administrations, applications, or dosages. The term can also include within its scope amounts effective to enhance or restore to substantially normal physiological function. In general, a “therapeutically effective amount” refers to an amount that is sufficient to achieve the desired therapeutic result or to have an effect on undesired symptoms but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

As used herein, the term “prophylactically effective amount” refers to an amount effective for preventing onset or initiation of a disease or condition.

As used herein, the term “effective amount” can refer to an amount of an inactive ingredient that is sufficient to achieve the desired modification of a physical property of the composition or material. For example, an “effective amount” of a binder refers to an amount that is sufficient to achieve the desired improvement in the property modulated by the formulation component. The specific level in terms of wt % in a composition required as an effective amount will depend upon a variety of factors including the amount and type of binder, amount and type of active ingredient, presence of other components in the formulation, and delivery mechanism of the pharmaceutical composition disclosed herein.

As used herein, the term “low dose,” as used herein, refers to the dose of a therapeutically effective amount of at least one substituted triphenylacetamide analog, e.g., 2,2-bis(4-fluorophenyl)-2-phenylacetamide, wherein said dose is at least about 10% lower than the clinically tested dose of 10 mg. The bioequivalence is established by comparing pharmacokinetic parameters, for example, AUC and Cₘₐₓ of the pharmaceutical composition of the present invention with a therapeutically effective amount of at least one substituted triphenylacetamide analog, e.g., 2,2-bis(4-fluorophenyl)-2-phenylacetamide, formulation in healthy human subjects in fed as well as fasting conditions.

As used herein, the term “AUC” refers to the area under the time/plasma concentration curve after administration of the pharmaceutical composition. AUC_{0-∞} denotes the area under the plasma concentration versus time curve from time 0 to infinity: AUC₀₋ₜ denotes the area under the plasma concentration versus time curve from time 0 to time t.

As used herein, the term “Cₘₐₓ” refers to the maximum concentration of a therapeutically effective amount of at least one substituted triphenylacetamide analog, e.g., 2,2-bis(4-fluorophenyl)-2-phenylacetamide, in the blood following administration of the pharmaceutical composition.

As used herein, the term “tₘₐₓ” refers to the time in hours when Cₘₐₓ is achieved following administration of the pharmaceutical composition.

As used herein, the term “D₁₀” refers to the particle size of a therapeutically effective amount of at least one substituted triphenylacetamide analog, e.g., 2,2-bis(4-fluorophenyl)-2-phenylacetamide, where 10% (w/v) of the particles have a size less than the defined D₁₀ value; “D” refers to the particle size of a therapeutically effective amount of at least one substituted triphenylacetamide analog, e.g., 2,2-bis(4-fluorophenyl)-2-phenylacetamide, where 50% (w/v) of the particles have a size less than the defined D₅₀ value; “D₉₀” refers to the particle size of a therapeutically effective amount of at least one substituted triphenylacetamide analog, e.g., 2,2-bis(4-fluorophenyl)-2-phenylacetamide, where 90% (w/v) of the particles have a size less than the defined D₉₀ value, when measured through Malvern Instrument Ltd.

As used herein, the term “stable” or “stability” of a therapeutic agent refers to chemical stability of the therapeutic agent, wherein not more than 1.5% w/w of total related substances are formed on storage at accelerated conditions of stability at 40° C. and 75% relative humidity or at 25° C. and 60% relative humidity for a period of at least six months or to the extent necessary for use of the composition.

As used herein, a “hematological disorder” refers to a disease or disorder that primarily affects blood and/or blood-forming organs. Hematological disorders can include genetic disorders such as, for example, sickle cell disease, thalassemia, methemoglobinemia, hereditary stomacytosis, and the like. It is understood that hematological disorders can further include anemias, myelodysplastic syndrome, myeloproliferative disorders, coagulopathies, hematological malignancies including, but not limited to, Hodgkin's disease, non-Hodgkin's lymphoma, multiple myeloma, and leukemias. Hematological disorders can also include hemochromatosis. In any of these aspects, the methods and pharmaceutical compositions disclosed herein can be useful for treating hematological disorders.

As used here, “abnormal cell proliferation” refers to a condition in which cell cycle regulation is disrupted such as that which occurs, for example, when a proto-oncogene, DNA repair gene, or tumor suppressor gene undergoes a mutation. In a further aspect, abnormal cell proliferation can lead to an accumulation of abnormal cell numbers and a condition such as, for example, cancer. In one aspect, the methods and compositions disclosed herein can be useful for treating diseases associated with abnormal cell proliferation including, but not limited to, bladder cancer, breast cancer, brain cancer, an endocrine cancer, retinoblastoma, cervical cancer, colon cancer, rectal cancer, endometrial cancer, renal cell carcinoma, renal pelvis carcinoma, Wilms tumor, a cancer of the oral cavity, liver cancer, gall bladder cancer, cholangiocarcinoma, melanoma, mesothelioma, myelodysplastic syndrome, acute myelogenous leukemia, non-small cell lung cancer, basal cell skin cancer, squamous cell skin cancer, ovarian cancer, pancreatic cancer, prostate cancer, soft tissue sarcoma, osteosarcoma, small cell lung cancer, thyroid cancer, other cancers, or a combination thereof.

As used herein, “area-under-the-curve value” describes drug concentration in blood plasma over time. In one aspect, drug concentration is measured at discrete time points and area-under-the-curve is estimated using the trapezoid rule (i.e., a technique for approximating the definite integral of a curve). In one aspect, area-under-the-curve value is useful for approximating bioavailability of a drug over time.

“Maximum plasma concentration” is the peak concentration of a pharmaceutical or drug achieved in the plasma of a subject to whom a drug has been administered. Maximum plasma concentration is typically measured after a first dose but before a second dose is administered.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents, and are meant to include future updates.

As used herein, “dose” or “dosage” can refer to physically discrete units suitable for use in a subject, each unit containing a predetermined quantity of a disclosed compound and/or a pharmaceutical composition thereof calculated to produce the desired response or responses in association with its administration. The dose can be administered in a single dosage or in multiple dosages, for example from 1 to 4 or more times per day. When multiple dosages are used, the amount of each dosage can be the same or different. Moreover, a dose comprising a first therapeutic agent and a second therapeutic agent can be in separate dosage forms or combined in a single dosage form.

As used herein, “single dosage form”, “single dose”, “unit dosage form”, “unit dose”, and “single dose form” can be used interchangeably and refer to a single drug administration entity combining at least one substituted triphenylacetamide analog. e.g., 2,2-bis(4-fluorophenyl)-2-phenylacetamide, in a therapeutically effective amount, optionally additional disclosed therapeutic agents, a pharmaceutically acceptable carrier, and other pharmaceutical acceptable excipients, inactive ingredients, and the like as disclosed herein. In various instances, the single dosage form can be a single tablet, capsule, or liquid. That is, “single dosage form” refers to a presentation form comprising a defined amount of at least one disclosed therapeutic agent, with the intention of applying the total of such amount as a single dosage. As an illustration, a representative single dosage of the present disclosure can be a tablet or a capsule comprising at least one substituted triphenylacetamide analog, e.g., 2,2-bis(4-fluorophenyl)-2-phenylacetamide, in an amount to provide a therapeutically effective dose as disclosed herein below. This aforementioned list of single dosage forms is not intended to be limiting in any way, but merely to represent typical examples of single dosage forms.

It is further understood that a single dosage form refers to physically discrete units suitable as unitary dosages, each unit containing a predetermined quantity of active ingredient calculated to produce the desired therapeutic effect in association with the required pharmaceutical carrier. That is, a “single dosage form” can be a single dose wherein ail active and inactive ingredients are combined in a suitable system, such that the patient or person administering the drug to the patient can open a single container or package with the entire dose contained therein. In some instances, a “single dosage form” can be a powder in a packet or container comprising a therapeutically effective amount of one or more therapeutic agents that can be mixed with a specified volume of liquid. Typical examples of unit dosage forms are tablets (including scored or coated tablets), capsules or pills for oral administration: powder packets; wafers; and segregated multiples thereof. This list of unit dosage forms is not intended to be limiting in any way, but merely to represent typical examples of unit dosage forms.

As used herein, “combined formulation” refers to the mixture of two or more isolated pharmaceutical compositions comprising, e.g., at least one substituted triphenylacetamide analog, e.g., 2,2-bis(4-fluorophenyl)-2-phenylacetamide, and a second therapeutic agent, such as, for example, a hydroxyurea analog, a DNMT1 inhibitor, a gene therapy agent, GBT440, luspatercept, a P-selectin binder such as crizanlizumab, a pyruvate kinase M2 activator, an iron chelator, an HOAC inhibitor, into a single dosage form.

As used herein, “combined administration” or “co-administration” refers to administration of two or more isolated pharmaceutical compositions, e.g., at least one substituted triphenylacetamide analog, e.g., 2,2-bis(4-fluorophenyl)-2-phenylacetamide, and a second therapeutic agent, such as, for example, pentoxifylline, pentosan polysulfate sodium, voxelotor, 5-hydroxymethyl-2-furfural (5-HMF), a hydroxyurea analog, a DNMT1 inhibitor, a gene therapy agent, luspatercept, a P-selectin binder such as crizanlizumab, a pyruvate kinase M2 activator, an iron chelator, an HDAC inhibitor, in separate dosage forms (e.g. separate pills) that can be taken together (simultaneous administration) or in a particular sequence (sequential administration).

As used herein; the terms “mixture” and “combination”, e.g., a combination therapeutic agent, can refer to multiple components or ingredients formed into one resulting component, e.g., a single dosage form comprising components that can be separate but contained in a single dosage form. A combination therapeutic is also inclusive of components that can be administered in the same treatment regimen even if not physically formed into a single component or contained in a single dosage form. As used herein, the terms “mixture” and “combination” may be used interchangeably.

The term “pharmaceutically acceptable” describes a material that is not biologically or otherwise undesirable, i.e., without causing an unacceptable level of undesirable biological effects or interacting in a deleterious manner.

The term “pharmaceutically acceptable carrier” is used herein to refer to a carrier that is useful in preparing a pharmaceutical composition that is generally safe, non-toxic and neither biologically nor otherwise-undesirable, and is acceptable for veterinary use as well as human pharmaceutical use. A “pharmaceutically acceptable carrier” as used in the specification and claims can include both one and more than one such carrier. By “pharmaceutically acceptable” it is meant the carrier must be compatible with the other ingredients of the formulation and not deleterious to the recipient thereof.

The term “pharmaceutically acceptable salts”, as used herein, means salts of the active principal agents which are prepared with acids or bases that are tolerated by a biological system or tolerated by a subject or tolerated by a biological system and tolerated by a subject when administered in a therapeutically effective amount. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include, but are not limited to; sodium, potassium, calcium, ammonium, organic amino, magnesium salt, lithium salt, strontium salt or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include, but are not limited to; those derived from inorganic acids like hydrochloric, hydrobromic, nitric, carbonic, monohydrogencarbonic, phosphoric, monohydrogen phosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like.

The term “pharmaceutically acceptable ester” refers to esters of compounds of the present disclosure which hydrolyze in vivo and include those that break down readily in the human body to leave the parent compound or a salt thereof. Examples of pharmaceutically acceptable, non-toxic esters of the present disclosure include C1 to C6 alkyl esters and C5 to C7 cycloalkyl esters, although C1 to C4 alkyl esters are preferred. Esters of disclosed compounds can be prepared according to conventional methods. Pharmaceutically acceptable esters can be appended onto hydroxy groups by reaction of the compound that contains the hydroxy group with acid and an alkylcarboxylic acid such as acetic acid, or with acid and an arylcarboxylic acid such as benzoic acid. In the case of compounds containing carboxylic acid groups, the pharmaceutically acceptable esters are prepared from compounds containing the carboxylic acid groups by reaction of the compound with base such as triethylamine and an alkyl halide, for example with methyl iodide, benzyl iodide, cyclopentyl iodide or alkyl triflate. They also can be prepared by reaction of the compound with an acid such as hydrochloric acid and an alcohol such as ethanol or methanol.

The term “pharmaceutically acceptable amide” refers to non-toxic amides of the present disclosure derived from ammonia, primary C1 to C6 alkyl amines and secondary C1 to C6 dialkyl amines. In the case of secondary amines, the amine can also be in the form of a 5- or 6-membered heterocycle containing one nitrogen atom. Amides derived from ammonia, C1 to C3 alkyl primary amides and C1 to C2 dialkyl secondary amides are preferred. Amides of disclosed compounds can be prepared according to conventional methods. Pharmaceutically acceptable amides can be prepared from compounds containing primary or secondary amine groups by reaction of the compound that contains the amino group with an alkyl anhydride, aryl anhydride, acyl halide, or aroyl halide. In the case of compounds containing carboxylic acid groups, the pharmaceutically acceptable amides are prepared from compounds containing the carboxylic acid groups by reaction of the compound with base such as triethylamine, a dehydrating agent such as dicyclohexyl carbodiimide or carbonyl diimidazole, and an alkyl amine, dialkylamine, for example with methylamine, diethylamine, and piperidine. They also can be prepared by reaction of the compound with an acid such as sulfuric acid and an alkylcarboxylic acid such as acetic acid, or with acid and an arylcarboxylic acid such as benzoic acid under dehydrating conditions such as with molecular sieves added. The composition can contain a compound of the present disclosure in the form of a pharmaceutically acceptable prodrug.

As used herein, “oral administration” or “orally” refers to the introduction of a pharmaceutical composition into a subject by way of the oral cavity (e.g., in liquid or solid form), e.g., a capsule or tablet, although other oral dosage forms are contemplated and disclosed herein. Oral administration is inclusive of dosage forms that are swallowed or ingested by the oral cavity, and transit, in some form, the gastro-intestinal tract such that therapeutic agents are absorbed, at least in part, from the gastro-intestinal tract. It is understood that oral administration is also inclusive of any mode of administration that is by way of the oral cavity, including, but not limited to, sublingual administration and buccal administration.

### Clinical Context

The major source of morbidity and mortality of patients suffering from sickle cell disease is vascular occlusion caused by the sickled cells, which causes repeated episodes of pain in both acute and chronic form and also causes ongoing organ damage with the passage of time. It has long been recognized and accepted that the deformation and distortion of sickle cell erythrocytes upon complete deoxygenation is caused by polymerization and intracellular gelation of sickle hemoglobin, hemoglobin S (Hb S). The phenomenon is well reviewed and discussed by Eaton et al., Blood 70:1245 (1987).

The intracellular gelatin and polymerization of Hb S can occur at any time during an erythrocyte's journey through the vasculature. Thus, erythrocytes in patients with sickle cell disease containing no polymerized hemoglobin S may pass through the microcirculation and return to the lungs without sickling, may sickle in the veins or may sickle in the capillaries.

The probability of each of these events is determined by the delay time for intracellular gelation relative to the appropriate capillary transit time (Eaton, et al., Blood 47: 621 (1976)). In turn, the delay time is dependent upon the oxygenation state of the hemoglobin, with deoxygenation shortening the delay time. If it is thermodynamically impossible for intracellular gelation to take place, or if the delay time at venous oxygen pressures is longer than about 15 seconds, cell sickling will not occur. If the delay time is between about 1 and 15 seconds, the red cell will likely sickle in the veins. If the delay time is less than about 1 second, red cells will sickle within the capillaries.

For red cells that sickle within the capillaries, a number of consequent events are possible. These range from no effect on transit time, to transient occlusion of the capillary, to a more permanent blockage that may ultimately result in ischemia or infarction of the surrounding cells, and in the subsequent destruction of the red cell.

Normal erythrocytes are comprised of approximately 70% water. Water crosses a normal erythrocyte membrane in milliseconds. Loss of cell water causes an exponential increase in cytoplasmic viscosity as the mean cell hemoglobin concentration (MCHC) rises above about 32 g/dl. Since cytoplasmic viscosity is a major determinate of erythrocyte deformability and sickling, the dehydration of the erythrocyte has substantial rheological and pathological consequences. Regulation of erythrocyte dehydration is recognized as an important therapeutic approach for treating sickle cell disease. Since cell water follows any osmotic change in intracellular ion concentration, maintaining the red cell's potassium concentration is of particular importance (Stuart et al., Brit J Haematol. 69:1-4 (1988)).

Many approaches to therapeutically treating dehydrated sickle cells (thus decreasing polymerization of hemoglobin S by lowering the osmolality of plasma) have been tried with limited success, including the following approaches: intravenous infusion of distilled water (Gye et al., Am. J Med. Sci. 266: 267-277(1973)); administration of the antidiuretic hormone vasopressin together with a high fluid intake and salt restriction (Rosa et al., M Eng. J Med. 303:1138-1143 (1980)); Charache et al., Blood 58: 892-896 (1981)); the use of monensin to increase the cation content of the sickle cell (Clark et al., J. Clin. Invest, 70:1074-1080 (1982)): Fahim et al., Life Sciences 29:1959-1966 (1981)); intravenous administration of cetiedil citrate (Benjamin et al., Blood 67: 1442-1447 (1986)); Berkowitz of al., Am. J. Hematol. 17: 217-223 (1984)); Stuart et al., J Clin. Pathol. 40:1182-1186 (1987)); and the use of oxpentifylline (Stuart et al., supra).

Another approach towards therapeutically treating dehydrated sickle cells involves altering erythrocyte potassium flux by targeting a calcium-dependent potassium channel (Ishi et al., Proc. Natl. Acad. Sci. 94(21): 11651-6 (1997)). This calcium activated potassium channel is also referred to as the Gardos channel (Brugnara et al, J. Clin. Invest. 92: 520-526 (1993)). Recently, a cloned human intermediate conductance calcium activated potassium channel, hIK.I, was shown to be substantially similar to the Gardos channel in terms of both its biophysical and pharmacological properties (Ishi, supra).

Methods that have been used to inhibit the Gardos channel include the administration to erythrocytes of imidazole, nitroimidazole and triazole antimycotic agents such as clotrimazole (U.S. Pat. No. 5,273,992 to Brugnara et al.). Clotrimazole, an imidazole-containing antimycotic agent, has been shown to be a specific, potent inhibitor of the Gardos channel of normal and sickle erythrocytes, and prevents Ca⁺²-dependent dehydration of sickle cells both in vitro and in vivo (Brugnara, supra; De Franceschi et al., J. Clin. Invest. 93: 1670-1676 (1994)). When combined with a compound which stabilizes the oxyconformation of Hb S, clotrimazole induces an additive reduction in the clogging rate of a micropore filter and may attenuate the formation of irreversibly sickled cells (Stuart et al., J. Haematol. 86:820-823 (1994)). Other compounds that contain a heteroaryl imidazole-like moiety believed to be useful in reducing sickle erythrocyte dehydration via Gardos channel inhibition include miconazole, econazole, butoconazole, oxiconazole and sulconazole. Although these compounds have been demonstrated to be effective at reducing sickle cell dehydration, other imidazole compounds have been found incapable of inhibiting the Gardos channel and preventing loss of potassium.

Since sickle cell anemia is a chronic disease, agents designed for treating it will ideally exhibit certain characteristics that are less essential in drugs for treating resolvable illnesses (e.g., fungal infections). A clinically useful Gardos channel inhibitor will exhibit extremely low toxicity over a prolonged course of administration, will have an excellent bioavailability, will be highly specific for the Gardos channel and will be potent in its interactions with this channel. Although clotrimazole and certain related compounds have been shown to inhibit the Gardos channel and prevent loss of potassium, these compounds are less than ideal clinical agents for the treatment of sickle cell anemia. Of primary concern is the fact that prolonged administration of imidazole antimycotics has been demonstrated to result in hepatotoxicity (see, for example, Rodriguez et al., Toxicology 6: 83-92 (1995); Finder et al., Medicina 58: 277-81 (1998); and Rodriguez et al., J Biochem. Toxicol. 11: 127-31 (1996)). The trend towards toxicity of an agent must be balanced with other characteristics such as its bioavailability, target selectivity and potency.

senicapoc is an ion-channel blocker that selectively blocks potassium efflux through the Gardos channel in red blood cells (Riles). Preclinical studies and studies in transgenic models of sickle cell disease have shown that senicapoc increases hemoglobin levels and decreases the density of cells and hemolysis. senicapoc is well tolerated when administered at a dose of 10 mg to sickle cell disease patients, producing a dose-dependent increase in hemoglobin and a decrease in markers of hemolysis. However, clinical studies in sickle cell disease were terminated due to a lack of efficacy and no significant improvement in the rate of sickle cell painful crises was observed in patients treated with senicapoc compared to those on placebo (Br J Haematol. 2011 April; 153(1):92-104). Additionally, senicapoc has demonstrated pharmacological activity against malaria, chronic asthma, liver disease and cancer.

Despite these promising pharmacological activities, senicapoc has limitations that limits clinical use. senicapoc is a very hydrophobic drug (log P 3.59) with poor aqueous solubility (975 μg/mL) and moderate oral bioavailability (51%). senicapoc has a half-life of 1 h in rats, with a maximum concentration attained after 4 h when administered orally. In recent years, lipid-based formulations to increase the solubility and oral bioavailability of poorly water-soluble compounds have received significant attention.

Very few publications and/or patents about the pharmaceutical composition of senicapoc are available. WO2019083454A1 mentions novel topical nanoliposomes to enhance the ocular bioavailability of senicapoc with improved the residence time by up to 12-fold that of the free drug (Phua et at, 2018), However, when applied to oral delivery, liposomes are characterized by several limitations, such as high cost, limited drug loading, poor scaling up and the use of organic solvents.

Buya, A. B. et al. (Intl J. Pharmaceut (2020) 580:119180) described a self-nanoemulsifying drug delivery system (SNEDDS) to improve the oral delivery of senicapoc. However, they did not disclose an underlying mechanism or provide any data supporting the senicapoc dose selection for human patients. Moreover, Buya, et al. failed to disclose a low dose formulation for senicapoc, either alone or in combination with an additional therapeutic agent. The skilled artisan can further appreciate that the SNEDDS formulation disclosed by Buya, et al, has several limitations, including: (a) use of ingredients with potential side-effects or toxic effects that could adversely affect patients; (b) limited accelerated stability; and (c) absence of site directed delivery of senicapoc to improve clinical efficacy in patients.

Several conventional processes have been described to increase the solubility and, hence, the bioavailability of poorly soluble pharmaceuticals or drugs. One such conventional process discloses the use of water-soluble high-molecular weight substances having low melting points, such as Carbowax, in combination with an insoluble drug. However, compositions prepared by this process possess poor redispersibility in water due to the low melting point and, therefore, are undesirable as pharmaceutical excipients.

Other methods of increasing the aqueous dissolution rate of poorly water-soluble drugs include the use of organic solvents to solubilize the poorly water-soluble drug or pharmaceutical composition. The use of organic solvents creates further problems with the health and safety aspects of organic solvents and the environmental unfriendliness and safety of organic solvents. All of these factors associated with the use of organic solvents considerably add to the cost of utilizing organic solvents in a method to increase the solubility of water-insoluble drugs as organic solvent recovery and containment devices are very costly. Other known surface active excipients can be affected by gastric pH or can be destructive to the intestinal mucosa.

In recent years, microemulsions, including self-microemulsifying drug delivery systems (“SMEDDS) have been extensively studied as a potential modality for oral drug delivery. Microemulsion systems contain a surfactant/co-surfactant blend which when added to a two-phase hydrophilic/lipophilic mixture, form a stable, optically clear, isotropic, colloidal system. The interest in the use of microemulsions as oral drug delivery systems stems from their ability to spontaneously form (emulsify) at a given temperature, their considerable solubilizing properties, the ability to be sterilized by filtration, and high physical stability. Another desirable feature of these mixtures is their ability to form a microemulsion when exposed to gastrointestinal fluids. This type of behavior makes SMEDDS good candidates for vehicles for the oral delivery of lipophilic or slightly water-soluble drugs.

Presently known Gardos channel inhibitors like senicapoc have low bioavailabilities. These deficiencies are of particular concern in conjunction with these drugs, as they must be regularly administered over a significant portion of a person's lifetime. With such drugs, patient compliance with the dosage regimen is crucial, and the simpler the regimen, the more likely a patient will comply with the regimen. Gardos channel inhibitors like senicapoc having low bioavailabilities must be frequently administered, raising the risk of missed doses and consequent plasma drug levels inadequate to prevent the dehydration of erythrocytes. In addition to frequent dosing, agents having low bioavailabilities must generally be administered in higher dosages than analogous agents with better bioavailabilities. At higher dosages, undesirable side effects and toxicity become a very real concern. Taken together, the low bioavailability of known Gardos channel inhibitors like senicapoc mandate higher and more frequent dosing, thereby increasing the risk of undesirable side effects and toxicity.

Therefore, reduction of senicapoc dose is highly beneficial. Therefore, there is a need to develop a composition of senicapoc which has a lower dose. The present inventors have developed an oral pharmaceutical composition of senicapoc, wherein said composition has enhanced bioavailability and lower dose. These advantages would lead to better patient compliance. Further, the present disclosure will enable to develop a senicapoc composition having low senicapoc to excipient ratio which would allow for it to be filled in a small capsule rather than a tablet. The smaller sized capsule would be advantageous for patients as it would be easier for swallowing.

Among other concerns, such a lifelong dosage regimen presents a serious risk of variable patient compliance with the regimen. If the dose level of the medication in a patient's system decreases as a result of poor compliance, this raises the risk of the occurrence of a sickle cell event and the concomitant pain and physical and physiological damage. Compounds having increased in vivo residence times and increased bioavailability allow for a simplified dosage regimen (i.e. fewer doses/day and/or less medication). Moreover, reducing the amount of compound administered carries with it the promise of reducing side effects resulting from the medication and/or its metabolites. Thus, it is highly desirable to provide formulations containing Gardos channel inhibitors like senicapoc that demonstrate good bioavailabilities and enhanced in vivo stabilities.

In view of the above-described shortcomings of currently known Gardos channel inhibitors like senicapoc a substantial advance in the treatment of sickle cell anemia is expected from the discovery of novel formulations of Gardos channel inhibitors that contain senicapoc, that are appreciably bioavailable, slowly metabolized and excreted and are easily administered as liquid or granules for children and patients who cannot swallow tablet medications, e.g., patients diagnosed with Alzheimer's disease or Parkinson's disease.

The present disclosure provides effective and suitable orally available pharmaceutical compositions to improve the solubility and cell permeation of Gardos channel inhibitors such as senicapoc, and methods of treating a disorder by administering same. In a further aspect, the disclosed pharmaceutical compositions are self-microemulsifying excipient formulations comprising an emulsion comprising an oil or other lipid material, a surfactant, and a hydrophilic co-surfactant that can increase the bioavailability of senicapoc. Also, in accordance with the present disclosure, methods are disclosed for making a drug delivery system for increasing the bioavailability of senicapoc by emulsifying senicapoc in combination with at least one additional drug with a self-microemulsifying excipient comprising an oil or other lipid material, a surfactant, and a hydrophilic co-surfactant and the drugs formulated thereby.

### Therapeutic Agents

The disclosed pharmaceutical compositions utilize disclosed therapeutic agents, alone and in various combinations as contemplated herein.

In one aspect, the pharmaceutical compositions disclosed herein comprise a therapeutic agent comprising at least one Gardos channel antagonist. In a further aspect, the at least one Gardos channel antagonist comprises at least one substituted triphenylacetamide analog, e.g., senicapoc. The Gardos channel is a calcium-activated potassium channel described by Gardos (Curr. Top. Membr. Transp. 10:217-277 (1978) and Nature 279:248-250 (1979)). Examples of Gardos channel antagonists include clotrimazole, triaryl methane derivatives, and triaryl carbonyl derivatives, e.g. triphenylacetamide derivatives such as 2,2-bis(4-fluorophenyl)-2-phenylacetamide.

In one aspect, the present disclosure relates to pharmaceutical compositions comprising at least one substituted triphenylacetamide analog, e.g., 2,2-bis(4-fluorophenyl)-2-phenylacetamide, having a structure represented by a formula:

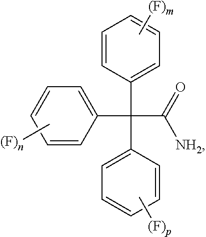

wherein m, n and p can be independently selected from 0 and 1 and at least one of m, n and p can be 1, or a pharmaceutically acceptable salt, solvate, or polymorph thereof; and a pharmaceutically acceptable carrier.

In one aspect, the present disclosure relates to pharmaceutical compositions comprising senicapoc, i.e., a comprising a compound having a structure represented by the formula:

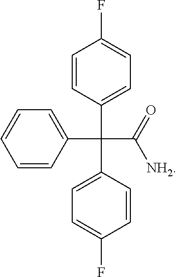 [SMILES: NC(=O)C(c1ccccc1)(c1ccc(F)cc1)c1ccc(F)cc1]

In one aspect, the disclosure relates to pharmaceutical compositions comprising a second therapeutic agent, such as, for example, pentoxifylline, pentosan polysulfate sodium, 5-hydroxymethyl-2-furfural (5-HMF), an HBS polymerization inhibitor, including, but not limited to, voxelotor (previously designated as GB1440 during pre-clinical and clinical research); a hydroxyurea analog; a DNMT1 inhibitor; a gene therapy agent; an erythroid maturation agent, including, but not limited to, luspatercept; a therapeutic agent that binds P-selectin (“a P-selectin binder”) such as monoclonal antibody such as crizanlizumab; a pyruvate kinase M2 activator (e.g., AG=3418), an iron chelator, and/or an HDAC inhibitor.

In various aspects, the DNMT1 inhibitor can be a nucleoside analog that is not a 2′-deoxycytidine analog such as 5-azacitidine (tradename: Vidaza), having the chemical name 4-amino-1-β-D-ribofuranosyl-s-triazin-2(1H)-one, having a structure represented by the formula immediately below.

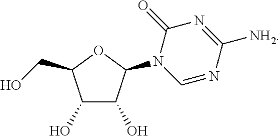 [SMILES: Nc1ncn([C@@H]2O[C@H](CO)[C@@H](O)[C@H]2O)c(=O)n1]

Azacitidine is believed to act by causing hypomethylation of DNA. The concentration of azacitidine required for maximum inhibition of DNA methylation in vitro does not cause major suppression of DNA synthesis.

In various aspects, the DNMT1 inhibitor can be a nucleoside analog that is not a 2′-deoxycytidine analog such as zebularine, also known as 1-(β-D-ribofuranosyl)-1,2-dihydropyrimidin-2-one or 2-pyrimidone-1-p-D-riboside, having a structure represented by the formula immediately below.

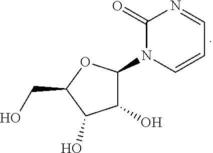 [SMILES: O=c1ncccn1[C@@H]1O[C@H](CO)[C@@H](O)[C@H]1O]

In a further aspect, a DNMT1 inhibitor is a non-nucleoside analogue, including, but not limited to, procainamide, procaine, hydralazine, RG108, and ((−)-epigallocatechin-3-gallate (EGCG).

In various aspects, the HDAC inhibitor can be any suitable HDAC inhibitor known to inhibit HDAC activity in a subject, including; but not limited to, suberoylanilide hydroxamic acid (SAHA, also marketed as Vorinostat), amide analogues of trichostatin A, hydroxamic acid analogs of trapoxin, and scriptaid (6-(1,3-Dioxo-1H, 3H-benzo[de]isoquinolin-2-yl)-hexanoic acid hydroxyamide) and analogs. In a further aspect, the HDAC inhibitor can be selected from the group consisting of suberoylanilide hydroxamic acid (SAHA), N-hydroxy-7-(4-dimethylaminobenzoyl)-aminoheptanamide (M344), N-hydroxy-8-(4-dimethylaminobenzoyl)-aminooctanamide (M360), N-hydroxy-6-(4-biphenylcarbonyl)-aminocapramide (M355), N-hydroxy-6-(4-dimethylaminobenzoylamino)-capramide (MD85), (S)-octanedioic acid hydroxyamide (1-phenethylcarbamoyl-2-phenyl-ethyl)-amide (SW68), (S)-octanedioic acid hydroxyamide (1-benzylcarbamoyl-2-phenyl-ethyl)-amide (SW70), (S)-3-(4-methoxyphenyl)-2-(7-hydroxycarbamoyl-heptanoylamino)-propionic acid methyl ester (SW99), (S)-2-(7-hydroxycarbamoyl-heptanoylamino)-3-thiophen-2-yl-propionic acid methyl ester (SW86), (S)-3-(4′-chlorobiphenyl-4-yl)-2-(7-hydroxycarbamoylheptanoylamino)-propionic acid methyl ester (SW183), (S)-3-(3′, 4′-dichlorobiphenyl-4-yl)-2-(7-hydroxycarbamoylheptanoylamino)-propionic acid methyl ester (SW187), (S)-2-(7-hydroxycarbamoylheptanoylamino)-3-(4-methoxybiphenyl-4-yl)-propionic acid methyl ester (SW188), (S)-2-(7-hydroxycarbamoylheptanoylamino)-3-(4′-methylbiphenyl-4-yl)-propionic acid methyl ester (SW189), (S)-3-(phenyl)-2-(7-hydroxycarbamoyl-heptanoylamino)-propionic acid methyl ester (M232), 6-(1,3-dioxo-1H,3H-benzo[de]isoquinolin-2-yl)hexanoic acid hydroxyamide (HR13), 6-(4,5,6,7-tetrachloro-1,3-dioxo-1,3-dihydroisoindol-2-yl) hexanoic acid hydroxyamide (HR10), 6-(1,3-dioxo-1,3-dihydroisoindol-2-0) hexanoic acid hydroxyamide (HR11), and combinations thereof.

In a further aspect, an iron chelator used as a second therapeutic agent can comprise one or more of deferoxamine, deferaslirox, deferprone, 1,10-phenanthroline, tagatose, zinc picolinate, chromium picolinate, deferitazole, sodium ferdetate, or pentetic acid.

### Pharmaceutical Compositions

In various aspects, the present disclosure relates to pharmaceutical compositions comprising a therapeutically effective amount of at least one substituted triphenylacetamide analog, e.g., 2,2-bis(4-fluorophenyl)-2-phenylacetamide, and at least one pharmaceutically acceptable excipient, providing enhanced bioavailability of the at least one substituted triphenylacetamide analog, thereby allowing low dosage forms of the at least one substituted triphenylacetamide analog to be administered to a subject.

In a further aspect, the present disclosure relates pharmaceutical compositions comprising a therapeutically effective amount of a first therapeutic agent comprising at least one substituted triphenylacetamide analog, e.g., 2,2-bis(4-fluorophenyl)-2-phenylacetamide, as disclosed herein, and optionally at least one second therapeutic agent as disclosed herein, and at least one pharmaceutically acceptable excipient. In a yet further aspect, the disclosed pharmaceutical compositions can be an orally available, low dose, fixed dose, delayed release combination product thus providing an easy-to-use product for a patient to use at home.

In a further aspect, the disclosed pharmaceutical compositions comprise a therapeutically effective amount of at least one substituted triphenylacetamide analog, e.g., 2,2-bis(4-fluorophenyl)-2-phenylacetamide, as disclosed herein and at least one pharmaceutically acceptable excipient, wherein the pharmaceutically acceptable excipient

In various aspects, the disclosed pharmaceutical compositions that enhance the oral bioavailability of at least one substituted triphenyl acetamide analog having a structure represented by a formula:

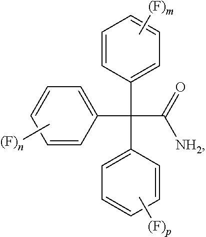

wherein m, n and p can be independently selected from 0 and 1 and at least one of m, n and p can be 1, or a pharmaceutically acceptable salt, solvate, or polymorph thereof, compared to conventionally formulations for substituted triphenyl acetamide analogs. In a further aspect, a disclosed pharmaceutical composition comprises an emulsion comprising at least one substituted triphenyl acetamide analog, an oil or lipid material, a surfactant, and a hydrophilic co-surfactant. In a still further aspect, the disclosed pharmaceutical composition is an emulsion wherein the emulsion is a microemulsion. In a yet further aspect, the disclosed pharmaceutical composition is an emulsion wherein the emulsion is self-emulsifying.

In various aspects, the present disclosure relates to pharmaceutical compositions comprising a therapeutically effective amount of at least one substituted triphenyl acetamide analog having a structure represented by a formula:

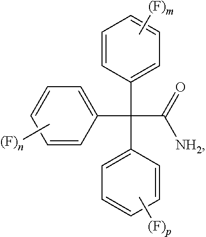

wherein m, n and p can be independently selected from 0 and 1 and at least one of m, n and p can be 1, or a pharmaceutically acceptable salt, solvate, or polymorph thereof; optionally one or more additional therapeutic agents; and a pharmaceutically acceptable carrier. In a further aspect, the least one substituted triphenyl acetamide analog has a structure represented by a formula:

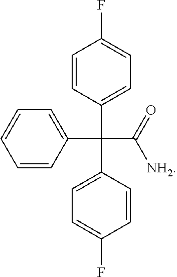 [SMILES: NC(=O)C(c1ccccc1)(c1ccc(F)cc1)c1ccc(F)cc1]

In various aspects, the present disclosure relates to pharmaceutical compositions comprising a therapeutically effective amount of at least one substituted triphenyl acetamide analog having a structure represented by a formula:

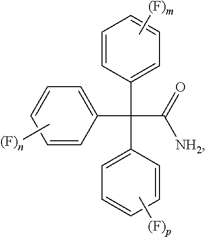

wherein m, n and p can be independently selected from 0 and 1 and at least one of m, n and p can be 1, or a pharmaceutically acceptable salt, solvate, or polymorph thereof; optionally one or more additional therapeutic agents; and a pharmaceutically acceptable carrier; wherein the pharmaceutically acceptable carrier comprises at least one oil or lipidic material, at least one surfactant, and at least one hydrophilic co-surfactant: and wherein the least one oil or lipidic material, the least one surfactant, and the least one hydrophilic co-surfactant form a suspension, an emulsion, or a microemulsion. In a further aspect, the foregoing pharmaceutical composition is a microemulsion. In a still further aspect, the emulsion is a self-emulsifying emulsion. In an even further aspect, the microemulsion is a self-emulsifying microemulsion.

In various aspects, the present disclosure relates to pharmaceutical compositions comprising a therapeutically effective amount of at least one substituted triphenyl acetamide analog having a structure represented by a formula:

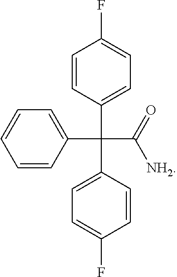 [SMILES: NC(=O)C(c1ccccc1)(c1ccc(F)cc1)c1ccc(F)cc1]

optionally one or more additional therapeutic agents; and a pharmaceutically acceptable carrier; wherein the pharmaceutically acceptable carrier comprises at least one oil or lipidic material, at least one surfactant, and at least one hydrophilic co-surfactant; and wherein the least one oil or lipidic material, the least one surfactant, and the least one hydrophilic co-surfactant form a suspension, an emulsion, or a microemulsion. In a further aspect, the foregoing pharmaceutical composition is a microemulsion. In a still further aspect, the microemulsion is a self-emulsifying microemulsion.

In various aspects, the present disclosure relates to pharmaceutical compositions formulated as a dosage form comprising a disclosed suspension, emulsion, or microemulsion within an enteric coating formulated as a capsule.

In various aspects, the present disclosure relates to pharmaceutical compositions comprising a therapeutically effective amount of at least one substituted triphenylacetamide analog, e.g., 2,2-bis(4-fluorophenyl)-2-phenylacetamide, as disclosed herein and at least one pharmaceutically acceptable excipient comprising at least two lipidic excipients. In a further aspect, the least two lipidic excipients comprise at least one hydrophilic excipient, i.e., having an HLB value greater than or equal to about 10, and at least one hydrophobic excipient. It is understood herein that “HLB” refers to the “hydrophilic-lipophilic balance”. The determination of an HLB value can be as known to the skilled artisan, e.g., as described by Griffin (J. Soc. Cosm. Chem. (1949) 1(5):311).

In various aspects, as discussed above, a disclosed pharmaceutical composition can comprise a therapeutically effective amount of at least one substituted triphenylacetamide analog, e.g., 2,2-bis(4-fluorophenyl)-2-phenylacetamide, as disclosed herein and at least one pharmaceutically acceptable excipient comprising at least two lipidic excipients. In a further aspect, the least two lipidic excipients comprise at least one hydrophilic excipient, i.e., having an HLB value greater than or equal to about 10, and at least one hydrophobic excipient. In a still further aspect, the least two lipidic excipients comprise at least one hydrophilic excipient, i.e., having an HLB value greater than or equal to about 10, and at least one hydrophobic excipient, having an HLB value less than or equal to about 10, in a yet further aspect, the least one hydrophilic excipient has an HLB value greater than or equal to about 10, about 11, about 12, about 13, about 14, or about 15; or a range comprising any two of the foregoing value; or any value within a range defined by the foregoing HLB values. In a still further aspect, the least one hydrophobic excipient has an HLB value less than or equal to about 10, about 9, about 8, about 7, about 6, or about 5; or a range comprising any two of the foregoing value; or any value within a range defined by the foregoing HLB values.

In a further aspect, the two lipidic excipients comprise hydrophilic excipient having a HLB value of at least 10, greater than 10, greater than or equal to 12, of between 12 and 14, and, the other lipidic excipient comprising a hydrophobic excipient, e.g., an oily vehicle. In a still further aspect, the disclosed pharmaceutical compositions comprise advantageously at least one hydrophilic excipient with an HLB value of at least 10 selected from the group consisting of glyceroyl macrogolglycerides, polyethyleneglycol derivatives, and mixtures thereof. In some instances, the disclosed pharmaceutical composition comprises from 20 to 80% by weight of hydrophilic excipient with an HLB value of at least 10 selected from the group consisting of glyceroyl macrogolglycerides, polyethyleneglycol derivatives, and mixtures thereof.

In a further aspect, the hydrophobic excipient, e.g., an oily vehicle, is selected from the group consisting of vegetable oils, medium chain triglycerides, fatty acid esters, amphiphilic oil, glycerol oleate derivative, and mixtures thereof. In a still further aspect, the disclosed pharmaceutical composition comprise from 5 to 70% by weight of an oily vehicle selected from the group consisting of vegetable oils, medium chain triglycerides, fatty acid esters, amphiphilic oil, glycerol oleate derivative, and mixtures thereof.

In various aspects, the disclosed pharmaceutical composition comprise a therapeutically effective amount of at least one substituted triphenylacetamide analog, e.g., 2,2-bis(4-fluorophenyl)-2-phenylacetamide, as disclosed herein and at least one pharmaceutically acceptable excipient comprising at least two lipidic excipients is a suspension, an emulsion, or a microemulsion. In a further aspect, the pharmaceutical composition is a suspension, an emulsion, or a microemulsion and comprises a therapeutically effective amount of at least one substituted triphenylacetamide analog, e.g., 2,2-bis(4-fluorophenyl)-2-phenylacetamide, as disclosed herein, and at least two lipidic excipients comprise at least one hydrophilic excipient, i.e., having an HLB value greater than or equal to about 10, and at least one hydrophobic excipient. In a still further aspect, the pharmaceutical composition is a suspension, an emulsion, or a microemulsion and comprises a therapeutically effective amount of at least one substituted triphenylacetamide analog, e.g., 2,2-bis(4-fluorophenyl)-2-phenylacetamide, as disclosed herein, at least one hydrophilic excipient, i.e., having an HLB value greater than or equal to about 10, and at least one hydrophobic excipient, having an HLB value less than or equal to about 10.

In a further aspect, the present disclosure relates to pharmaceutical compositions comprising a therapeutically effective amount of at least one substituted triphenylacetamide analog, e.g., 2,2-bis(4-fluorophenyl)-2-phenylacetamide, optionally at least one additional therapeutic agent, or a pharmaceutically acceptable salt thereof, and a pharmaceutically acceptable carrier. As used herein, “pharmaceutically-acceptable carriers” means one or more of a pharmaceutically acceptable diluents, preservatives, antioxidants, solubilizers, emulsifiers, coloring agents, releasing agents, coating agents, sweetening, flavoring and perfuming agents, and adjuvants. The disclosed pharmaceutical compositions can be conveniently presented in unit dosage form and prepared by any of the methods well known in the art of pharmacy and pharmaceutical sciences.

In a further aspect, the disclosed pharmaceutical compositions comprise a therapeutically effective amount of at least one substituted triphenylacetamide analog, e.g., 2,2-bis(4-fluorophenyl)-2-phenylacetamide, optionally at least one additional therapeutic agent, or a pharmaceutically acceptable salt thereof, a pharmaceutically acceptable carrier, and optionally one or more adjuvant. The disclosed pharmaceutical compositions include those formulated to allow administration orally.

In various aspects, the present disclosure also relates to a pharmaceutical composition comprising a pharmaceutically acceptable carrier and/or diluent and, as active ingredient, a therapeutically effective amount of at least one substituted triphenylacetamide analog, e.g., 2,2-bis(4-fluorophenyl)-2-phenylacetamide, or a pharmaceutically acceptable salt, a hydrate thereof, a solvate thereof, a polymorph thereof, or a stereochemically isomeric form thereof, optionally at least one additional therapeutic agent, or a pharmaceutically acceptable salt, a hydrate thereof, a solvate thereof, a polymorph thereof, or a stereochemically isomeric form thereof. In a further aspect, the at least one substituted triphenylacetamide analog, e.g., 2,2-bis(4-fluorophenyl)-2-phenylacetamide, or a pharmaceutically acceptable salt, a hydrate thereof, a solvate thereof, a polymorph thereof, or a stereochemically isomeric form thereof, optionally at least one additional therapeutic agent, or a pharmaceutically acceptable salt, a hydrate thereof, a solvate thereof, a polymorph thereof, or a stereochemically isomeric form thereof, may be formulated into various oral dosage forms for administration purposes, e.g., a capsule, a tablet, a delayed release formulation, and the like.

In a further aspect, the disclosed pharmaceutical composition comprise an excipient selected from the group consisting of Capryol™ 90, Capryol PGMC, Lauroglycol™ 90, Lauroglycol™ FCC, Labrafac™ PG, Labrafil® M1944CS, Labrafil®M 2125CS, Labrafil® M2130CS, Capmul® MCM, Miglyol 810, Miglyol 812, Lecithin (PL 90), Tween 80/Polysorbate 80, Tween 20/polysorbate 20, Lutrol® F 68, Lutrol® F 127, Labrasol®, Geluciore® 44/14, Geluciore® 50/13, Vitamin E-TPGS, Solutol® HS 15, Cremophor® EL, Cremophore® ELP, Cremophor® RH 40, Transcutol® P, Transcutol® HP, Soluphor® P, Pharmasolve V. Glycofurol, PEG 200-400, Propylene glycol, Soluplus®, long chain triglycerides (“LCTs”; including, but not limited to, soyabean oil, corn oil, safflower oil, and fish oil), cyclodextrins (“CD”), and combinations thereof.

In a further aspect, the disclosed pharmaceutical composition comprise an excipient selected from the group consisting of Vitamin E, PEG-DSPE, cholesterol, Gelucire 44/14, Hydrogenated castor oil 60, sodium dodecyl sulfate, tricaproin (oil), egg PC, Tween 80, Tricaproin (oil), Tween 80, Captex 355, Capmul MCM, Cremophor EL, absolute ethanol, captex 355, capmul MCM, Cremophor® EL, soybean oil, Maisine 35-1, lecithin, glycerin, poloxamer 338/Pluronic F108, soybean phosphatidyl choline, Solutol HS15, PEG 400, ethanol, miglyol 810N (MGT), egg lecithin, glycerol, propylene glycol, ethyl laurate, oleic acid, Cremophor® RH 40, Labrasol, ethyl oleate dextran 40, Carbitol, Capryol 90, Lauroglycol 90, Labrasolc, Transcutol, Labrafil 1944 CS, TPGS, Acconon E, Softigen 767, Inwitor 742, Transcutol P, Labrafil M1944 CS, SBEβCD, Eudragit EPO, Tween 20. Soluphor P, DRAB, lecithin, Precirol ATO 5f, Gelucire 50/3, and combinations thereof.

In a further aspect, the disclosed pharmaceutical compositions comprise at least one surfactant, preferably selected from the group consisting of sorbitan fatty acid esters, polysorbate derivatives, polyoxyethylene sorbitan fatty acid esters, sodium laurylsulphate, derivatives of lecithine, propylene glycol esters, fatty acid esters of propylene glycol, fatty acid esters of glycerol, polyethylene glycol, and mixtures thereof. For example, the composition contains from 1 to 10% by weight of at least one surfactant.

In a further aspect, the disclosed pharmaceutical compositions further comprise at least one disintegrant, preferably selected from the group consisting of povidone derivative, sodium croscarmellose and mixtures thereof.

In a further aspect, the disclosed pharmaceutical compositions comprise one or more surfactants and/or one or more disintegrants.

In some instances, an oil phase of the disclosed self-microemulsifying pharmaceutical compositions comprise lipid or glycerides comprising compounds such as GELUCIRE, Gattefosse Corporation, Westwood, N.J.), but can also include other suitable oil phase compounds for example, digestable or non-digestable oils and fats such as olive oil, corn oil, soybean oil, cottonseed oil, palm oil, and animal fats. Suitable surfactants or emulsifying agents used in the self-microemulsifying formulation of the present disclosure include LABRAFAC CM 10, a mixture of saturated C8-C10 polyglycalysed glycerides (HLB=10, Gattefosse Corporation, Westwood, N.J.) and other suitable surfactants, for example, long alkyl chain sulfonates/sulfates such as sodium dodecylbenzene sulfonate, sodium lauryl sulfate, and dialkyl sodium sulfosuccinate, quaternary ammonium salts, fatty alcohols such as lauryl, cetyl, and steryl, glycerylesters, fatty acid esters, and polyoxyethylene derivatives thereof.

Co-surfactants suitable for use with the disclosed self-emulsifying pharmaceutical compositions of the present disclosure are can be hydrophilic in nature. In various aspects, co-surfactants utilized in the present disclosure should possess an HLB number of greater than 8 based on the HLB system on which is well known to those skilled in the art. The HLB number provides a means for ranking surfactants based on the balance between the hydrophilic and lipophilic portions of the surfactant or emulsifying agent. That is, the higher the HLB number, the more hydrophilic the surfactant or emulsifying agent. In the present disclosure, the hydrophilic co-surfactant has a hydrophilic-lipophilic balance (HLB) of greater than 8. Typically, surfactants or emulsifiers within HLB in the range of 8-18 form oil/water emulsions. In the present disclosure, the preferred HLB range for the hydrophilic co-surfactant is between approximately 10 and 14. Additionally, hydrophilic co-surfactants utilized in the present disclosure are preferably alcohols of intermediate chain length such as hexanol, pentanol, and octanol which are known to reduce the oil/water interface and allow the spontaneous formulation of the emulsion. Preferred hydrophilic co-surfactants utilized in accordance with the present disclosure include LABRASOL, (Gattefosse Corporation, Westwood, N.J.), which is comprised of medium chain triglycerides derived from coconut oil having an HLB of 14 as well, as other co-surfactants having an HLB of greater than 8 such as lauryl alcohol.

The disclosed self-microemulsifying pharmaceutical compositions can further comprise an aqueous solvent such as triacetin, an acetylated derivative of glycerol, i.e., glyceryl triacetate or other suitable solvents. Triacetin is suitable since it is miscible in the oil/lipid phase and can be used to solubilize a hydrophobic drug. Additional materials and/or compounds can be added to alter the consistency of the emulsion. This may be done to increase the stability or emolliency of the emulsion. Such materials can include tragacanth, cetyl alcohol, stearic acid, and/or beeswax (Remington's Pharmaceutical Sciences, 1975).

The disclosed self-microemulsifying pharmaceutical compositions can be prepared by solubilizing the substituted triphenyl acetamide analog in a mixture of surfactant, co-surfactant and solvent. The oil phase can then be suitably prepared, if necessary, by heating or other preparatory means and can then be added to the solubilized drug formulation and thoroughly mixed. The emulsion can then be added to a suitable dosage form such as soft or hard-filled gelatin capsules and allowed to cool.

The relative proportions of surfactant and co-surfactant in the disclosed self-microemulsifying pharmaceutical compositions can influence the solubilizing and dissolution properties of the formulation. In general, the range of concentration of the surfactant/co-surfactant broadly ranges from 15 to 90% (v/v) and more preferably ranges from approximately from 45 to 55% (v/v). The concentration of the co-surfactant broadly ranges from 16 to 89% (v/v) and more preferably ranges from 30 to 40% (v/v). The relative amounts of surfactant to co-surfactant in the formulation of the present disclosure range from approximately 45 to 50% (v/v) with the preferred range being approximately 25 to 35% (v/v). Generally, the ratio of surfactant to co-surfactant ranges from approximately 1:2 to 1:3 depending on the properties of the surfactant and/or the co-surfactant.

The substituted triphenyl acetamide analog utilized in accordance with the present disclosure can further comprise an additional therapeutic agent, including, but not limited to, GST440, Pentosan Polysulfate Sodium, 5-hydroxymethyl-2-furfural (5-HMF). Other pharmaceutical ingredients or other drugs which are lipophilic or poorly water-soluble can also be used in accordance with the present disclosure. This list is not meant to be exhaustive, but rather provide examples of suitable compounds may be used in accordance with the present disclosure.

The disclosed pharmaceutical compositions comprise semi-solid dosage form comprising at least one substituted triphenylacetamide analog, e.g., 2,2-bis(4-fluorophenyl)-2-phenylacetamide, that advantageously provide improved bioavailability thereof. A semi-solid dosage form containing at least one substituted triphenylacetamide analog, e.g., 2,2-bis(4-fluorophenyl)-2-phenylacetamide, is a form in which the at least one substituted triphenylacetamide analog is mixed with suitable melted excipients. The molten mix is then filled for example into hard gelatine capsules or other pharmaceutically acceptable capsules. At ambient temperature (temperature for example of less than 20° C.), the content of the capsule is solid while at temperature higher than 20° C. (for example at temperature greater or equal to 30° C., advantageously greater or equal to 35° C., preferably substantially at body temperature, i.e., 37° C.+/−5° C.), it is liquid or semi-solid (paste). The at least one substituted triphenylacetamide analog may be solubilized in the mix of excipients or partially solubilized. The active ingredient may also be formulated as a suspension, emulsion or microemulsion. Various lipidic excipients are available to the formulator to obtain a semi-solid formulation. Excipients compatible with hard gelatin capsule shells are lipophilic liquid vehicles (refined specialty oils, medium-chain triglycerides and related esters), semi-solid lipophilic vehicles, solubilizing agents, emulsifying agents and absorption enhancers. The classification of fatty excipients is based on the hydrophilicity or lipophilicity of the excipients, characterized by the hydrophilic/lipophilic balance value (HLB). Examples of lipophilic excipients are vegetable oils (peanut oil, olive oil, soyabean oil, and the like), fatty adds (stearic acid, palmitic add, and the like), and/or fatty alcohols. Examples of hydrophilic excipients are polyethyleneglycol (PEG) with a molecular weight superior to 3,000. Examples of amphiphilic excipients are Poloxamers, Lecithin, PEG esters (Gelucire®).

The manufacturing advantages of the disclosed semi-solid formulations comprising at least one substituted triphenylacetamide analog, e.g., 2,2-bis(4-fluorophenyl)-2-phenylacetamide, include protection of the active ingredient from air and humidity; increasing the dissolution rate of the active ingredient, and hence of the bioavailability; diminution of the risk of contamination of the operator; diminution of the risk of cross contamination; no possibility of demixing under the effect of vibrational mixing during manufacturing process; and improved production process. The choice of the nature of the formulation of course influenced the stability of the pharmaceutical form and the bioavailability of the at least one substituted triphenylacetamide analog, e.g., 2,2-bis(4-fluorophenyl)-2-phenylacetamide, contained in it. Generally, a maximum bioavailability is achieved by preparing and keeping the drug in the amorphous/solubilized state in a solid dispersion or in a lipid-based formulation. Moreover, the disclosed pharmaceutical compositions can further provide diminished loss of insoluble crystalline forms during the dissolution/release following administration in vivo.

In various aspects, the disclosed pharmaceutical compositions can be a suspension, emulsion, microemulsion, self-emulsifying drug delivery systems (SEDDS) or self-emulsifying microemulsion drug delivery system (SMEDDS). The skilled artisan can appreciate that microemulsions can be advantageous compared to suspensions, such as emulsions and dispersions, for various reasons, including enhanced thermodynamic stability, lower energy input during manufacturing processes, and generally a longer shelf-life.

In a further aspect, the disclosed pharmaceutical compositions can be an oil-in-water (O/W) and water-in-oil (W/O) microemulsions comprising an oil, a surfactant, a cosurfactant, water and electrolytes.

In various aspects, the disclosed pharmaceutical compositions comprise a pharmaceutically acceptable capsule comprising at least one semi-solid composition of the disclosure, for example at least one composition of the invention as disclosed hereabove. The capsule can be selected from the group consisting of hard gelatine capsules, soft gelatine capsules, hypromellose capsules, starch capsules.

In various aspects, the disclosed pharmaceutical compositions comprise a therapeutically effective amount of at least one substituted triphenylacetamide analog, e.g., 2,2-bis(4-fluorophenyl)-2-phenylacetamide, and a pharmaceutically acceptable excipient, wherein said composition, when administered orally, provides an equivalent efficacy at a lower dose of in comparison to the clinically tested dose of 10-30 mg, wherein said dose is at least 20% lower, at least 15% lower, at least 10%, or at least 5% lower

In another aspect, the present disclosure relates to a pharmaceutical composition for oral administration comprising: (a) a therapeutically effective amount of at least one substituted triphenylacetamide analog, e.g., 2,2-bis(4-fluorophenyl)-2-phenylacetamide; and (b) an oily vehicle.

In another aspect of any of the above aspects, the pharmaceutical composition for oral administration comprises: (a) a therapeutically effective amount of at least one substituted triphenylacetamide analog, e.g., 2,2-bis(4-fluorophenyl)-2-phenylaectamide, in an amount of about 5.0 w/w to about 15 w/w based on the total weight; (b) an oily vehicle, and (c) surfactant in amount of about 0.05% w/w to about 10% w/w based on the total weight.

In one aspect of any of the above aspects, the pharmaceutical composition for oral administration comprises a therapeutically effective amount of at least one substituted triphenylacetamide analog, e.g., 2,2-bis(4-fluorophenyl)-2-phenylacetamide, in an amount of about 1 mg to 5 mg, 1 mg to 10 mg, 5 mg to 10 mg, 2 mg to 8 mg, 1 mg to 100 mg, 5 mg to 50 mg, 10 mg to 40 mg, 9 mg to 36 mg, or 8 mg to 32 mg; or a sub-range within any of the foregoing ranges; or any value or set of values within the foregoing ranges.

In another aspect of any of the above aspects, the pharmaceutical composition for oral administration comprises a therapeutically effective amount of at least one substituted triphenylacetamide analog, e.g., 2,2-bis(4-fluorophenyl)-2-phenylacetamide, in an amount of about 2 mg, 4 mg, 6 mg, 8 mg. 10 mg, 16 mg, 20 mg, 24 mg, 28 mg, or 32 mg.

In another aspect of any of the above aspects, the pharmaceutical composition for oral administration comprises a therapeutically effective amount of at least one substituted triphenylacetamide analog, e.g., 2,2-bis(4-fluorophenyl)-2-phenylacetamide, in an amount of about 6 mg/day.

In another aspect of any of the above aspects, the pharmaceutical composition for oral administration comprises in capsule a semi-solid suspension comprising a therapeutically effective amount of at least one substituted triphenylacetamide analog, e.g., 2,2-bis(4-fluorophenyl)-2-phenylacetamide in an amount, i.e., dose/capsule, of about 5 mg to about 7.0 mg, 5 mg to 8.0 mg, about 6.5 mg to about 9.0 mg, about 6.5 mg to 10 mg, about 13 mg to about 18 mg, about 13 mg to 20 mg, about 16.25 mg to about 22.5 mg, about 16.25 mg to 25 mg, about 19.5 mg to about 27, about 19.5 mg to mg 30 mg, about 22.75 mg to about 31.5 mg, about 22.75 mg to 35 mg, about 26 mg to about 36 mg, about 26 mg to 40 mg; or a sub-range within any of the foregoing ranges; or any value or set of values within the foregoing ranges.

In another aspect of any of the above aspects, the pharmaceutical composition for oral administration is in the form of a dispersion which is further filled into capsules.

In another aspect of the above aspects, the oily vehicle includes, but is not limited to, groundnut oil, olive oil, soybean oil, kernel oil, almond oil, safflower oil, sunflower oil, palm oil, sesame oil, canola oil, corn oil, castor oil, coconut oil, cotton seed oil, grape seed oil, and mixtures thereof.

In another aspect of any of the above aspects, the oily vehicle is present in an amount of about 1% w/w to about 99% w/w by the total weight of the composition; preferably in an amount of about 10% w/w to about 95% w/w by the total weight of the composition.

In another aspect of any of the above aspects, the oily vehicle is present in an amount of about 71% w/w to about 95% w/w by the total weight of the composition.

In another aspect of any of the above aspects, the ratio of a therapeutically effective amount of at least one substituted triphenylacetamide analog, e.g., 2,2-bis(4-fluorophenyl)-2-phenylacetamide, to the oily vehicle ranges from about 1:99 to about 99:1.

In another aspect of any of the above aspects, the pharmaceutical composition for oral administration further comprises a surfactant.

In another aspect of any of the above aspects, the surfactant includes, but is not limited to, anionic, cationic, or non-ionic surfactants; sorbitan fatty acid esters; polysorbates prepared from lauric, palmitic, stearic, and oleic acids; mononylphenyl ethers of polyethylene glycols such as nanoxynols; polyoxyethylene monoesters such as polyoxyethylethylene monostearate, polyoxyethylene monolaurate, and polyoxyethylene monooleate; dioctyl sodium sulfosuccinate; sodium lauryl sulphate; lecithin; fatty acid esters of propylene glycol; fatty acid esters of glycerol; poloxamers; and mixtures thereof.

In another aspect of any of the above aspects, the surfactant is present in an amount of about 0.05% w/w to about 10.0% w/w by the total weight of the composition.

In yet another aspect of any of the above aspects, the pharmaceutical composition for oral administration further comprises other excipients like antioxidants, preservatives, and alkaline stabilizers.

In yet another aspect of any of the above aspects, the pharmaceutical composition for oral administration is substantially free of wax.

The term “substantially free of wax” as used herein means that the pharmaceutical composition for oral administration contains wax less than about 10% by weight of the composition, less than about 9% by weight, less than about 8% by weight, or less than about 7% by weight.

In yet another aspect of any of the above aspects, the pharmaceutical composition for oral administration is free of wax.

In yet another aspect of any of the above aspects, the pharmaceutical composition for oral administration comprises a therapeutically effective amount of at least one substituted triphenylacetamide analog, e.g., 2,2-bis(4-fluorophenyl)-2-phenylacetamide, having a particle size distribution such that the D₉₀ is less than 60 μm, less than 55 μm, less than 50 μm, less than 45 μm, less than 40 μm, less than 35 μm, less than 30 μm, less than 25 μm, less than 20 μm, less than 15 μm, or less than 10 μm.

In yet another aspect of any of the above aspects, the pharmaceutical composition for oral administration comprises a therapeutically effective amount of at least one substituted triphenylacetamide analog, e.g., 2,2-bis(4-fluorophenyl)-2-phenylacetamide, having a particle size distribution such that the D₉₀ is less than 30 μm.

In another aspect of any of the above aspects, the pharmaceutical composition for oral administration comprises a therapeutically effective amount of at least one substituted triphenylacetamide analog, e.g., 2,2-bis(4-fluorophenyl)-2-phenylacetamide, having a particle size distribution such that the D₅₀ is less than 40 μm, less than 35 μm, less than 30 μm, less than 25 μm, less than 20 μm, less than 15 μm, less than 10 μm, or less than 5 μm.

In yet another aspect of any of the above aspects, the pharmaceutical composition for oral administration comprises a therapeutically effective amount of at least one substituted triphenylacetamide analog, e.g., 2,2-bis(4-fluorophenyl)-2-phenylacetamide, having a particle size distribution such that the D₅₀ is less than 15 μm.

In another aspect of any of the above aspects, the pharmaceutical composition for oral administration comprises a therapeutically effective amount of at least one substituted triphenylacetamide analog, e.g., 2,2-bis(4-fluorophenyl)-2-phenylacetamide, having a particle size distribution such that the D₁₀ is less than 20 μm, less than 18 μm, less than 17 μm, less than 15 μm, less than 12 μm, less than 10 μm, less than 8 μm, less than 7 μm, less than 5 μm, or less than 2 μm.

In yet another aspect of any of the above aspects, the pharmaceutical composition for oral administration comprises a therapeutically effective amount of at least one substituted triphenylacetamide analog, e.g., 2,2-bis(4-fluorophenyl)-2-phenylacetamide, having a particle size distribution such that the Ow is less than 7 μm.

In yet another aspect of any of the above aspects, the pharmaceutical composition for oral administration comprises a therapeutically effective amount of at least one substituted triphenylacetamide analog, e.g., 2,2-bis(4-fluorophenyl)-2-phenylacetamide, having a particle size distribution that the D₉₀ is less than 60 μm, the D₉₀ is less than 50 μm, or the D₅₀ is less than 40 μm.

In yet another aspect of any of the above aspects, the pharmaceutical composition for oral administration comprises a therapeutically effective amount of at least one substituted triphenylacetamide analog, e.g., 2,2-bis(4-fluorophenyl)-2-phenylacetamide, having a particle size distribution such that the D₉₀ is less than 60 μm, Dais less than 40 μm, D₅₀ is less than 30 μm, or D₁₀ is less than 20 μm.

In yet another aspect of any of the above aspects, the pharmaceutical composition for oral administration comprises a therapeutically effective amount of at least one substituted triphenylacetamide analog, e.g., 2,2-bis(4-fluorophenyl)-2-phenylacetamide, having a particle size distribution such that the D₉₀ is less than 30 μm, Do is less than 15 μm, or D₁₀ is less than 10 μm.

In yet another aspect of any of the above aspects, the pharmaceutical composition for oral administration comprises a therapeutically effective amount of at least one substituted triphenylacetamide analog, e.g., 2,2-bis(4-fluorophenyl)-2-phenylacetamide, having particle size distribution of a therapeutically effective amount of at least one substituted triphenylacetamide analog, e.g., 2,2-bis(4-fluorophenyl)-2-phenylacetamide, is such that the ratio of D₉₀/D₅₀ is about 1.5-2.5 μm, D₅₀/D₁₀ is about 1.8 to 3.3 and D₅₀/D₁₀ is about 3.0 to 6.1.

In another aspect, the pharmaceutical composition for oral administration comprising a therapeutically effective amount of at least one substituted triphenylacetamide analog, e.g., 2,2-bis(4-fluorophenyl)-2-phenylacetamide, and a pharmaceutically acceptable excipient when stored at a temperature 20° C. and 75% relative humidity is stable for a period of six months.

In one aspect of any of the above aspects, the pharmaceutical composition for oral administration is filled in a capsule having a fill occupancy more than 40%.

In another aspect of any of the above aspects, the pharmaceutical composition for oral administration in the form of dispersion is filled in hard gelatin capsule.

In another aspect of any of the above aspects, the pharmaceutical composition for oral administration is filled in a capsule with use of the “Lidose® technology” to provide a formulation of a therapeutically effective amount of at least one substituted triphenylacetamide analog, e.g., 2,2-bis(4-fluorophenyl)-2-phenylacetamide, with enhanced bioavailability.

In another aspect, the pharmaceutical composition for oral administration comprising a therapeutically effective amount of at least one substituted triphenylacetamide analog, e.g., 2,2-bis(4-fluorophenyl)-2-phenylacetamide, and a pharmaceutically acceptable excipient wherein said composition is filled in a following capsule size: Dose/Capsule Capsule size 5 mg Size 2 or smaller, 6 mg Size 2 or smaller, 8 mg Size 2 or smaller, 10 mg Size 1 or smaller, 16 mg Size 1 or smaller, 20 mg Size 0 or smaller, 24 mg Size 0 or smaller, 28 mg Size 00 or smaller, or 32 mg Size 00 or smaller.

In one aspect of any of the above aspects, the pharmaceutical composition for oral administration has a therapeutically effective amount of at least one substituted triphenylacetamide analog, e.g., 2,2-bis(4-fluorophenyl)-2-phenylacetamide, to excipient ratio 1:5 to 1:18.

In another aspect of any of the above aspects, the pharmaceutical composition for oral administration has fill weight of less than 600 mg (excluding capsule shell weight).

In another aspect, there is provided a process for the preparation of a low dose oral pharmaceutical composition comprising a therapeutically effective amount of at least one substituted triphenylacetamide analog. e.g., 2,2-bis(4-fluorophenyl)-2-phenylacetamide, and an oily vehicle wherein the process comprises: (a) dispersing a therapeutically effective amount of at least one substituted triphenylacetamide analog, e.g., 2,2-bis(4-fluorophenyl)-2-phenylacetamide, in an oily carrier; (b) milling the dispersion to get the desired particle size; (c) adding one or more excipients to the above dispersion; (d) optionally adding an oily carrier to the dispersion of step (c); and (e) filling the dispersion into capsules.

In another aspect, there is provided a process for the preparation of a low dose oral pharmaceutical composition comprising a therapeutically effective amount of at least one substituted triphenylacetamide analog, e.g., 2,2-bis(4-fluorophenyl)-2-phenylacetamide, and an oily vehicle wherein the process comprises: (a) dispersing a therapeutically effective amount of at least one substituted triphenylacetamide analog, e.g., 2,2-bis(4-fluorophenyl)-2-phenylacetamide, in an oily carrier; (b) milling the dispersion to get the desired particle size; (c) adding one or more excipients to the above dispersion; (d) adding an oily carrier to the dispersion of step (c); and (e) filling the dispersion into capsules.

In one aspect of any of the above aspects, the oily carrier used in step (a) is present in an amount which is at least 25% w/w of the total amount of the oily carrier.

In another aspect of any of the above aspects, the composition has a density of 0.8 to 1.1 g/mL

In another aspect, the pharmaceutical composition for oral administration comprises a therapeutically effective amount of at least one substituted triphenylacetamide analog, e.g., 2,2-bis(4-fluorophenyl)-2-phenylacetamide, and a pharmaceutically acceptable excipient, wherein said composition has an a therapeutically effective amount of at least one substituted triphenylacetamide analog, e.g., 2,2-bis(4-fluorophenyl)-2-phenylacetamide, to excipient ratio of about 1:5 to about 1:18 and releases not less than 50% of a therapeutically effective amount of at least one substituted triphenylacetamide analog, e.g., 2,2-bis(4-fluorophenyl)-2-phenylacetamide, in 15 minutes when measured in United States Pharmacopeia (USP) type II dissolution apparatus, paddle at 75 rpm, in 900 mL of pH 7.4 phosphate buffer with 70 mg/L Pancreatin and 4.5% lauryldimethylamine oxide (as 30% solution) with spiral coated sinker.

In one aspect of any of the above aspects, the pharmaceutical composition for oral administration releases not less than about 10%, about 20%, about 40%, about 60%, about 85% in 30 minutes.

In another aspect of any of the above aspects, the pharmaceutical composition for oral administration has viscosity of about 50 to 450 cps at room temperature. The viscosity was measured by Brookfield Viscometer, spindle number S-02 stirred at 20 RPM.

In another aspect, the pharmaceutical composition for oral administration comprises a therapeutically effective amount of at least one substituted triphenylacetamide analog, e.g., 2,2-bis(4-fluorophenyl)-2-phenylacetamide, and a pharmaceutically acceptable excipient, wherein said composition releases not less than 50% of a therapeutically effective amount of at least one substituted triphenylacetamide analog, e.g., 2,2-bis(4-fluorophenyl)-2-phenylacetamide, in 15 minutes when measured in United States Pharmacopeia (USP) type II dissolution apparatus, paddle at 75 rpm, in 900 mL of borate Buffer (pH 8.0) containing 0.5% cetrimide and 50 mg/L of pancreatin (with alternate sinkers).

Examples of suitable antioxidants include, but are not limited to, butylated hydroxyl anisole, butylated hydroxyl toluene, tocopherol, ascorbyl palmitate, ascorbic acid, sodium metabisulfite, sodium sulfite, sodium thiosulfate, propyl gallate, and mixtures thereof. The antioxidant is present in an amount of about 0.002% w/w to about 2% w/w of the total composition.

Examples of alkaline stabilizers include, but are not limited to, sodium hydroxide, potassium hydroxide, sodium carbonate or bicarbonate, potassium carbonate or bicarbonate, lithium hydroxide, triethylamine, meglumine, methylamine, and mixtures thereof.

Examples of suitable preservatives include, but are not limited to, methyl paraben, ethyl paraben, propyl paraben, butyl paraben, benzoic acid, sodium benzoate, benzyl alcohol, sorbic acid, potassium sorbate, and mixtures thereof.

The size reduction of a therapeutically effective amount of at least one substituted triphenylacetamide analog, e.g., 2,2-bis(4-fluorophenyl)-2-phenylacetamide, is achieved by wet milling the dispersion of a therapeutically effective amount of at least one substituted triphenylacetamide analog, e.g., 2,2-bis(4-fluorophenyl)-2-phenylacetamide, in an oily vehicle using mechanical means such as a jet mill, ball mill, or media mills such as a sand mill. DYNO®-mill, or a bead mill. The grinding media in these mills can comprise spherical particles such as stainless steel beads or zirconium oxide balls.

Pharmaceutical compositions of the present disclosure encompass any composition made by admixing the active ingredients and a pharmaceutically acceptable carrier. The carrier may take a wide variety of forms depending on the form of preparation desired for administration, e.g., oral administration. Thus, the pharmaceutical composition of the disclosure can be presented as discrete units suitable for oral administration such as capsules, cachets or tablets each containing a predetermined amount of the active ingredients. Further, the composition can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the composition may also be administered by controlled release means and/or delivery devices. The foregoing list is illustrative only and is not intended to be limiting in any way.

Pharmaceutically acceptable salts can be prepared from pharmaceutically acceptable non-toxic bases or acids. For therapeutic use, salts of the disclosed therapeutic agents are those wherein the counter ion is pharmaceutically acceptable. However, salts of acids and bases which are non-pharmaceutically acceptable may also find use, for example, in the preparation or purification of a pharmaceutically acceptable compound. All salts, whether pharmaceutically acceptable or not, are contemplated by the present disclosure. Pharmaceutically acceptable acid and base addition salts are meant to comprise the therapeutically active non-toxic acid and base addition salt forms which the disclosed compounds are able to form.

In various aspects, a disclosed therapeutic agent comprising an acidic group or moiety, e.g., a carboxylic acid group, can be used to prepare a pharmaceutically acceptable salt. For example, such a disclosed therapeutic agent may comprise an isolation step comprising treatment with a suitable inorganic or organic base. In some cases, it may be desirable in practice to initially isolate a compound from the reaction mixture as a pharmaceutically unacceptable salt and then simply convert the latter back to the free acid compound by treatment with an acidic reagent, and subsequently convert the free add to a pharmaceutically acceptable base addition salt. These base addition salts can be readily prepared using conventional techniques, e.g., by treating the corresponding acidic compounds with an aqueous solution containing the desired pharmacologically acceptable cations and then evaporating the resulting solution to dryness, preferably under reduced pressure. Alternatively, they also can be prepared by mixing lower alkanolic solutions of the acidic compounds and the desired alkali metal alkoxide together, and then evaporating the resulting solution to dryness in the same manner as before.

Bases which can be used to prepare the pharmaceutically acceptable base-addition salts of the base therapeutic agents are those which can form non-toxic base-addition salts, i.e., salts containing pharmacologically acceptable cations such as, alkali metal cations (e.g., lithium, potassium and sodium), alkaline earth metal cations (e.g., calcium and magnesium), ammonium or other water-soluble amine addition salts such as N-methylglucamine-(meglumine), lower alkanolammonium and other such bases of organic amines. In a further aspect, derived from pharmaceutically acceptable organic non-toxic bases include primary, secondary, and tertiary amines, as well as cyclic amines and substituted amines such as naturally occurring and synthesized substituted amines. In various aspects, such pharmaceutically acceptable organic non-toxic bases include, but are not limited to, ammonia, methylamine, ethylamine, propylamine, isopropylamine, any of the four butylamine isomers, betaine, caffeine, choline, dimethylamine, diethylamine, diethanolamine, dipropylamine, diisopropylamine, di-n-butylamine, N,N′-dibenzylethylenediamine, pyrrolidine, piperidine, morpholine, trimethylamine, triethylamine, tripropylamine, tromethamine, 2-diethylaminoethanol, 2-dimethylaminoethanol, ethanolamine, quinuclidine, pyridine, quinoline and isoquinoline; benzathine, N-methyl-D-glucamine, ethylenediamine, N-ethylmorpholine, N-ethylpiperidine, glucamine, glucosamine, methylglucamine, morpholine, piperazine, piperidine, polyamine resins, procaine, purines, theobromine, hydrabamine salts, and salts with amino acids such as, for example, histidine, arginine, lysine and the like. The foregoing salt forms can be converted by treatment with acid back into the free acid form.

In various aspects, a disclosed therapeutic agent comprising a protonatable group or moiety, e.g., an amino group, can be used to prepare a pharmaceutically acceptable salt. For example, such a disclosed therapeutic agent may comprise an isolation step comprising treatment with a suitable inorganic or organic acid. In some cases, it may be desirable in practice to initially isolate a compound from the reaction mixture as a pharmaceutically unacceptable salt and then simply convert the latter back to the free base compound by treatment with a basic reagent, and subsequently convert the free base to a pharmaceutically acceptable acid addition salt. These acid addition salts can be readily prepared using conventional techniques, e.g., by treating the corresponding basic compounds with an aqueous solution containing the desired pharmacologically acceptable anions and then evaporating the resulting solution to dryness, preferably under reduced pressure. Alternatively, they also can be prepared by treating the free base form of the disclosed compound with a suitable pharmaceutically acceptable non-toxic inorganic or organic acid.

Acids which can be used to prepare the pharmaceutically acceptable acid-addition salts of the base therapeutic agent are those which can form non-toxic acid-addition salts, i.e., salts containing pharmacologically acceptable anions formed from their corresponding inorganic and organic adds. Exemplary, but non-limiting, inorganic acids include hydrochloric hydrobromic, sulfuric, nitric, phosphoric and the like. Exemplary, but non-limiting, organic acids include acetic, benzenesulfonic, benzoic, camphorsulfonic, citric, ethanesulfonic, fumaric, gluconic, glutamic, isethionic, lactic, maleic, malic, mandelicmethanesulfonic, mucic, pamoic, pantothenic, succinic, tartaric, p-toluenesulfonic acid and the like. In a further aspect, the acid-addition salt comprises an anion formed from hydrobromic, hydrochloric, maleic, phosphoric, sulfuric, and tartaric acids.

In practice, the therapeutic agents of the present disclosure, or pharmaceutically acceptable salts thereof, of the present disclosure can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the pharmaceutical compositions of the present disclosure can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the present disclosure, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

It is especially advantageous to formulate the disclosed pharmaceutical compositions in a single dosage form for ease of administration and uniformity of dosage.

The pharmaceutical compositions disclosed herein comprise a therapeutic agent of the present disclosure (or pharmaceutically acceptable salts thereof) as an active ingredient, a pharmaceutically acceptable carrier, and optionally one or more additional therapeutic agents. In various aspects, the disclosed pharmaceutical compositions can include a pharmaceutically acceptable carrier and a disclosed compound, or a pharmaceutically acceptable salt thereof. In a further aspect, a disclosed compound, or pharmaceutically acceptable salt thereof, can also be included in a pharmaceutical composition in combination with one or more other therapeutically active compounds. The instant compositions include compositions suitable for oral administration, although the most suitable route in any given case will depend on the particular host, and nature and seventy of the conditions for which the active ingredient is being administered. The pharmaceutical compositions can be conveniently presented in unit dosage form and prepared by any of the methods well known in the art of pharmacy.

Pharmaceutical compositions of the present disclosure may be prepared by any of the methods well known in the art of pharmacy. Techniques and compositions for making dosage forms useful for materials and methods described herein are described, for example, in the following references: Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Gandertan, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Harwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.).

The therapeutic agents described herein are typically to be administered in admixture with suitable pharmaceutical diluents, excipients, extenders, or carriers (termed herein as a pharmaceutically acceptable carrier, or a carrier) suitably selected with respect to the intended form of administration and as consistent with conventional pharmaceutical practices. The deliverable compound will be in a form suitable for oral, rectal, topical, intravenous injection or parenteral administration. Carriers include solids or liquids, and the type of carrier is chosen based on the type of administration being used. The compounds may be administered as a dosage that has a known quantity of the compound.

Because of the ease in administration, oral administration can be a preferred dosage form for the disclosed pharmaceutical compositions, and tablets and capsules represent the most advantageous oral dosage unit forms in which case solid pharmaceutical carriers are obviously employed. However, other dosage forms may be suitable depending upon clinical population (e.g., age and severity of clinical condition), solubility properties of the specific disclosed compound used, and the like. Accordingly, the disclosed compounds can be used in oral dosage forms such as pills, powders, granules, elixirs, tinctures, suspensions, syrups, and emulsions. In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques.

The disclosed pharmaceutical compositions in an oral dosage form can comprise one or more pharmaceutical excipient and/or additive. Non-limiting examples of suitable excipients and additives include gelatin, natural sugars such as raw sugar or lactose, lecithin, pectin, starches (for example corn starch or amylose), dextran, polyvinyl pyrrolidone, polyvinyl acetate, gum arabic, alginic acid, tylose, talcum, lycopodium, silica gel (for example colloidal), cellulose, cellulose derivatives (for example cellulose ethers in which the cellulose hydroxy groups are partially etherified with lower saturated aliphatic alcohols and/or lower saturated, aliphatic oxyalcohols, for example methyl oxypropyl cellulose, methyl cellulose, hydroxypropyl methyl cellulose, hydroxypropyl methyl cellulose phthalate), fatty acids as well as magnesium, calcium or aluminum salts of fatty acids with 12 to 22 carbon atoms, in particular saturated (for example stearates), emulsifiers, oils and fats, in particular vegetable (for example, peanut oil, castor oil, olive oil, sesame oil, cottonseed oil, corn oil, wheat germ oil, sunflower seed oil, cod liver oil, in each case also optionally hydrated); glycerol esters and polyglycerol esters of saturated fatty acids C₁₂H₂₄O₂ to C₁₈H₃₆O₂ and their mixtures, it being possible for the glycerol hydroxy groups to be totally or also only partly esterified (for example mono-, di- and triglycerides); pharmaceutically acceptable mono- or multivalent alcohols and polyglycols such as polyethylene glycol and derivatives thereof, esters of aliphatic saturated or unsaturated fatty acids (2 to 22 carbon atoms, in particular 10-18 carbon atoms) with monovalent aliphatic alcohols (1 to 20 carbon atoms) or multivalent alcohols such as glycols, glycerol, diethylene glycol, pentacrythritol, sorbitol, mannitol and the like, which may optionally also be etherified, esters of citric acid with primary alcohols, acetic acid, urea, benzyl benzoate, dioxolanes, glyceroformals, tetrahydrofurfuryl alcohol, polyglycol ethers with C1-C12-alcohols, dimethylacetamide, lactamides, lactates, ethylcarbonates, silicones (in particular medium-viscous polydimethyl siloxanes), calcium carbonate, sodium carbonate, calcium phosphate, sodium phosphate, magnesium carbonate and the like. In some aspects, the pharmaceutically acceptable excipient can be mannitol, microcrystalline cellulose, crospovidone, magnesium stearate, another excipient as disclosed herein, or a combination thereof.

Other auxiliary substances useful in preparing an oral dosage form are those which cause disintegration (so-called disintegrants), such as: cross-linked polyvinyl pyrrolidone, sodium carboxymethyl starch, sodium carboxymethyl cellulose or microcrystalline cellulose. Conventional coating substances may also be used to produce the oral dosage form. Those that may for example be considered are: polymerizates as well as copolymerizates of acrylic acid and/or methacrylic acid and/or their esters: copolymerizates of acrylic and methacrylic acid esters with a lower ammonium group content (for example EudragitR RS), copolymerizates of acrylic and methacrylic acid esters and trimethyl ammonium methacrylate (for example EudragitR RL); polyvinyl acetate; fats, oils, waxes, fatty alcohols; hydroxypropyl methyl cellulose phthalate or acetate succinate; cellulose acetate phthalate, starch acetate phthalate as well as polyvinyl acetate phthalate, carboxy methyl cellulose; methyl cellulose phthalate, methyl cellulose succinate, -phthalate succinate as well as methyl cellulose phthalic acid half ester; zein; ethyl cellulose as well as ethyl cellulose succinate; shellac, gluten; ethylcarboxyethyl cellulose; ethacrylate-maleic acid anhydride copolymer; maleic acid anhydride-vinyl methyl ether copolymer; styrol-maleic acid copolymerizate; 2-ethyl-hexyl-acrylate maleic acid anhydride; crotonic acid-vinyl acetate copolymer; glutaminic acid/glutamic acid ester copolymer; carboxymethylethylcellulose glycerol monooctanoate; cellulose acetate succinate; polyarginine.

Plasticizing agents that may be considered as coating substances in the disclosed oral dosage forms are; citric and tartaric acid esters (acetyl-triethyl citrate, acetyl tributyl-, tributyl-, triethyl-citrate); glycerol and glycerol esters (glycerol diacetate, -triacetate, acetylated monoglycerides, castor oil); phthalic acid esters (dibutyl-, diamyl-, diethyl-, dimethyl-, dipropyl-phthalate), di-(2-methoxy- or 2-ethoxyethyl)-phthalate, ethylphthalyl glycolate, butylphthalylethyl glycolate and butylglycolate; alcohols (propylene glycol, polyethylene glycol of various chain lengths), adipates (diethyladipate, di-(2-methoxy- or 2-ethoxyethyl)-adipate; benzophenone; diethyl- and diburylsebacate, dibutylsuccinate, dibutyltartrate; diethylene glycol dipropionate; ethyleneglycol diacetate, -dibutyrate, -dipropionate; tributyl phosphate, tributyrin; polyethylene glycol sorbitan monooleate (polysorbates such as Polysorbar 50); sorbitan monooleate.

Moreover, suitable binders, lubricants, disintegrating agents, coloring agents, flavoring agents, flow-inducing agents, and melting agents may be included as carriers. The pharmaceutical carrier employed can be, for example, a solid, liquid, or gas. Examples of solid carriers include, but are not limited to, lactose, terra alba, sucrose, glucose, methylcellulose, dicalcium phosphate, calcium sulfate, mannitol, sorbitol talc, starch, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In various aspects, a binder can include, for example, starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. In a further aspect, a disintegrator can include, for example, starch, methyl cellulose, agar, bentonite, xanthan gum, and the like.

In various aspects, an oral dosage form, such as a solid dosage form, can comprise a disclosed therapeutic agent that is attached to polymers as targetable drug carriers or as a prodrug. Suitable biodegradable polymers useful in achieving controlled release of a drug include, for example, polylactic acid, polyglycolic acid, copolymers of polylactic and polyglycolic acid, caprolactones, polyhydroxy butyric acid, polyorthoesters, polyacetals, polydihydropyrans, polycyanoacylates, and hydrogels, preferably covalently crosslinked hydrogels.

Tablets may contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period.

A tablet containing a disclosed compound can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

In various aspects, a solid oral dosage form, such as a tablet, can be coated with an enteric coating to prevent ready decomposition in the stomach. In various aspects, enteric coating agents include, but are not limited to, hydroxypropylmethylcellulose phthalate, methacrylic add-methacrylic acid ester copolymer, polyvinyl acetate-phthalate and cellulose acetate phthalate. Akihiko Hasegawa “Application of solid dispersions of Nifedipine with enteric coating agent to prepare a sustained-release dosage form” Chem. Pharm. Bull. 331615-1619 (1985). Various enteric coating materials may be selected on the basis of testing to achieve an enteric coated dosage form designed ab initio to have a preferable combination of dissolution time, coating thicknesses and diametral crushing strength (e.g., see S. C. Porter et al. “The Properties of Enteric Tablet Coatings Made From Polyvinyl Acetate-phthalate and Cellulose acetate Phthalate”, J. Pharm. Pharmacol. 22:42p (1970)). In a further aspect, the enteric coating may comprise hydroxypropyl-methylcellulose phthalate, methacrylic acid-methacrylic acid ester copolymer, polyvinyl acetate-phthalate and cellulose acetate phthalate.

In various aspects, an oral dosage form can be a solid dispersion with a water soluble or a water insoluble carrier. Examples of water soluble or water insoluble carrier include, but are not limited to, polyethylene glycol, polyvinylpyrrolidone, hydroxypropylmethyl-cellulose, phosphatidylcholine, polyoxyethylene hydrogenated castor oil, hydroxypropylmethylcellulose phthalate, carboxymethylethylcellulose, or hydroxypropylmethylcellulose, ethyl cellulose, or stearic acid.

In various aspects, an oral dosage form can be in a liquid dosage form, including those that are ingested, or alternatively, administered as a mouth wash or gargle. For example, a liquid dosage form can include aqueous suspensions, which contain the active materials in admixture with excipients suitable for the manufacture of aqueous suspensions. In addition, oily suspensions may be formulated by suspending the active ingredient in a vegetable oil, for example arachis oil, olive oil, sesame oil or coconut oil, or in a mineral oil such as liquid paraffin. Oily suspensions may also contain various excipients. The pharmaceutical compositions of the present disclosure may also be in the form of oil-in-water emulsions, which may also contain excipients such as sweetening and flavoring agents.

For the preparation of solutions or suspensions it is, for example, possible to use water, particularly sterile water, or physiologically acceptable organic solvents, such as alcohols (ethanol, propanol, isopropanol. 1,2-propylene glycol, polyglycols and their derivatives, fatty alcohols, partial esters of glycerol), oils (for example peanut oil, olive oil, sesame oil, almond oil, sunflower oil, soya bean oil, castor oil, bovine hoof oil), paraffins, dimethyl sulphoxide, triglycerides and the like.

In the case of a liquid dosage form such as a drinkable solutions, the following substances may be used as stabilizers or solubilizers: lower aliphatic mono- and multivalent alcohols with 2-4 carbon atoms, such as ethanol, n-propanol, glycerol, polyethylene glycols with molecular weights between 200-600 (for example 1 to 40% aqueous solution), diethylene glycol monoethyl ether, 1,2-propylene glycol, organic amides, for example amides of aliphatic C1-C6-carboxylic acids with ammonia or primary, secondary or tertiary C1-C4-amines or C1-C4-hydroxy amines such as urea, urethane, acetamide, N-methyl acetamide, N,N-diethyl acetamide, N,N-dimethyl acetamide, lower aliphatic amines and diamines with 2-6 carbon atoms, such as ethylene diamine, hydroxyethyl theophylline, tromethamine (for example as 0.1 to 20% aqueous solution), aliphatic amino acids.

In preparing the disclosed liquid dosage form can comprise solubilizers and emulsifiers such as the following non-limiting examples can be used: polyvinyl pyrrolidone, sorbitan fatty acid esters such as sorbitan trioleate, phosphatides such as lecithin, acacia, tragacanth, polyoxyethylated sorbitan monooleate and other ethoxylated fatty acid esters of sorbitan, polyoxyethylated fats, polyoxyethylated oleotriglycerides, linolizated oleotriglycerides, polyethylene oxide condensation products of fatty alcohols, alkylphenols or fatty acids or also 1-methyl-3-(2-hydroxyethyl)imidazolidone-(2). In this context, polyoxyethylated means that the substances in question contain polyoxyethylene chains, the degree of polymerization of which generally lies between 2 and 40 and in particular between 10 and 20. Polyoxyethylated substances of this kind may for example be obtained by reaction of hydroxyl group-containing compounds (for example mono- or diglycerides or unsaturated compounds such as those containing oleic acid radicals) with ethylene oxide (for example 40 Mol ethylene oxide per 1 Mol glyceride). Examples of oleotriglycerides are olive oil, peanut oil, castor oil, sesame oil, cottonseed oil, corn oil. See also Dr. H. P. Fiedler “Lexikon der Hillsstoffe fir Pharmazie, Kostnetik und angrenzende Gebiete” 1971, pages 191-195.

In various aspects, a liquid dosage form can further comprise preservatives, stabilizers, buffer substances, flavor correcting agents, sweeteners, colorants, antioxidants and complex formers and the like. Complex formers which may be for example be considered are: chelate formers such as ethylene diamine retrascetic acid, nitrilotriacetic acid, diethylene triamine pentacetic acid and their salts.

It may optionally be necessary to stabilize a liquid dosage form with physiologically acceptable bases or buffers to a pH range of approximately 6 to 9. Preference may be given to as neutral or weakly basic a pH value as possible (up to pH 8).

In order to enhance the solubility and/or the stability of a disclosed compound in a disclosed liquid dosage form, a parenteral injection form, or an intravenous injectable form, it can be advantageous to employ α-, β- or γ-cyclodextrins or their derivatives, in particular hydroxyalkyl substituted cyclodextrins, e.g. 2-hydroxypropyl-β-cyclodextrin or sulfobutyl-β-cyclodextrin. Also co-solvents such as alcohols may improve the solubility and/or the stability of the compounds according to the present disclosure in pharmaceutical compositions.

In various aspects, a disclosed liquid dosage form, a parenteral injection form, or an intravenous injectable form can further comprise liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Pharmaceutical compositions containing a compound of the present disclosure, and/or pharmaceutically acceptable salts thereof, can also be prepared in powder or liquid concentrate form.

The pharmaceutical composition (or formulation) may be packaged in a variety of ways. Generally, an article for distribution includes a container that contains the pharmaceutical composition in an appropriate form. Suitable containers are well known to those skilled in the art and include materials such as bottles (plastic and glass), sachets, foil blister packs, and the like. The container may also include a tamper proof assemblage to prevent indiscreet access to the contents of the package. In addition, the container typically has deposited thereon a label that describes the contents of the container and any appropriate warnings or instructions.

The disclosed pharmaceutical compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Pharmaceutical compositions comprising a disclosed compound formulated in a compatible pharmaceutical carrier may also be prepared, placed in an appropriate container, and labeled for treatment of an indicated condition.

The exact dosage and frequency of administration depends on the particular disclosed compound, a product of a disclosed method of making, a pharmaceutically acceptable salt, solvate, or polymorph thereof, a hydrate thereof, a solvate thereof, a polymorph thereof, or a stereochemically isomeric form thereof; the particular condition being treated and the severity of the condition being treated; various factors specific to the medical history of the subject to whom the dosage is administered such as the age: weight, sex, extent of disorder and general physical condition of the particular subject, as well as other medication the individual may be taking; as is welt known to those skilled in the art. Furthermore, it is evident that said effective daily amount may be lowered or increased depending on the response of the treated subject and/or depending on the evaluation of the physician prescribing the compounds of the present disclosure.

In another aspect, the pharmaceutical composition further includes a coating. In some aspects, the coating is an enteric coating. In still another aspect, the coating can be a sugar coating, a film coating, a compression coating, or a combination thereof. In aspects where the coating is a film coating, it can be a cellulose ether polymer such as, for example, a hydroxypropyl methylcellulose, hydroxypropyl cellulose, or methylcellulose polymer or a combination thereof.

Depending on the particular dosage form, a disclosed pharmaceutical composition can comprise from 0.05 to 99% by weight, preferably from 0.1 to 70% by weight, more preferably from 0.1 to 50% by weight of the active ingredient, and, from 1 to 99.95% by weight, preferably from 30 to 99.9% by weight, more preferably from 50 to 99.9% by weight of a pharmaceutically acceptable carrier, all percentages being based on the total weight of the composition.

In a further aspect, the disclosed pharmaceutical composition has a unit dose form comprising from about 0.01 to 1000 mg per kg patient body weight per day of a disclosed therapeutic agent and can be administered in single or multiple doses. In various aspects, the dosage level of a disclosed therapeutic agent will be about 0.1 to about 500 mg/kg per day, about 0.1 to 250 mg/kg per day, or about 0.5 to 100 mg/kg per day. A suitable dosage level of a disclosed therapeutic agent can be about 0.01 to 1000 mg/kg per day, about 0.01 to 500 mg/kg per day, about 0.01 to 250 mg/kg per day, about 0.05 to 100 mg/kg per day, or about 0.1 to 50 mg/kg per day. Within this range the dosage can be 0.05 to 0.5, 0.5 to 5.0 or 5.0 to 50 mg/kg per day. For oral administration, the disclosed pharmaceutical compositions can be provided in the form of tablets containing a disclosed therapeutic agent of from about 1.0 to about 1000 mg of the active ingredient, particularly 1.0, 5.0, 10, 15, 20, 25, 50, 75, 100, 150, 200, 250, 300, 400, 500, 600, 750, 800, 900 and 1000 mg of the active ingredient for the symptomatic adjustment of the dosage of the patient to be treated. In one aspect, the tablets contain 40, 400, or 1000 mg of a disclosed therapeutic agent. The unit dose form can be administered on a regimen of 1 to 4 times per day, preferably once or twice per day. The unit dose form can be adjusted to provide the optimal therapeutic response. In a still further aspect, the unit dose form is administered once per week or is administered 2-3 times per week.

Disclosed unit doses as described can be administered more than once a day, for example, 2, 3, 4, 5 or 6 times a day. In various aspects, such unit doses can be administered 1 or 2 times per day, so that the total dosage for a 70 kg adult is in the range of 0.001 to about 15 mg per kg weight of subject per administration. In a further aspect, dosage is 0.01 to about 1.5 mg per kg weight of subject per administration, and such therapy can extend for a number of weeks or months, and in some cases, years, it will be understood, however, that the specific dose level for any particular subject will depend on a variety of factors including the activity of the specific compound employed; the age, body weight, general health, sex and diet of the individual being treated; the time and route of administration; the rate of excretion; other drugs that have previously been administered; and the severity of the particular disease undergoing therapy, as is well understood by those of skill in the area.

A typical unit dose can be taken once a day, or can be taken multiple times per day, or can be one time-release capsule or tablet taken once a day and containing a proportionally higher content of active ingredient. Alternatively, the unit dose can be taken once per week or 2-3 times per week. The time-release effect can be obtained by capsule materials that dissolve at different pH values, by capsules that release slowly by osmotic pressure, or by any other known means of controlled release.

In a further aspect, the disclosed pharmaceutical composition has a unit dose form comprising at least one substituted triphenylacetamide analog, e.g., 2,2-bis(4-fluorophenyl)-2-phenylacetamide, from about 1 to about 60 mg, or at least one substituted triphenylacetamide analog, e.g., 2,2-bis(4-fluorophenyl)-2-phenylacetamide, from about 1.5 to about 15 mg, or at least one substituted triphenylacetamide analog, e.g., 2,2-bis(4-fluorophenyl)-2-phenylacetamide, in an amount of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or about 60 mg, or a combination of any of the foregoing values, or a range encompassing any of the foregoing values. In a still further aspect, the foregoing pharmaceutical composition is formulated for oral administration.

a further aspect, the disclosed pharmaceutical composition has a unit dose form comprising at least one substituted triphenylacetamide analog, e.g., 2,2-bis(4-fluorophenyl)-2-phenylacetamide, of from about 1 to 600 mg per m² patient body surface area per day and can be administered in single or multiple doses. In various aspects, the dosage level will be about 1 to about 500 mg/m² per day, about 1 to 250 mg/m² per day, or about 1 to 150 mg/m² per day. A suitable dosage level can be about 1 to 600 mg/m² per day, about 1 to 250 mg/m² per day, about 1 to 150 mg/m² per day, about 1 to 100 mg/m² per day, or about 1 to 50 mg/m² per day. Within this range the dosage can be 1 to 50, 50 to 100, 100 to 150, 150 to 250, or 250 to 500 mg/m² per day. In a still further aspect, the disclosed pharmaceutical composition has a unit dose form comprising at least one substituted triphenylacetamide analog, e.g., 2,2-bis(4-fluorophenyl)-2-phenylacetamide, of from about 5 mg/m² to about 136 mg/m², or can be about 5, 10, 15, 20, 25, 30, 35, 40, 45, 50, 55, 60, 65, 70, 75, 80, 85, 90, 95, 100, 105, 110, 115, 120, 125, 130, or about 135 mg/m², or a combination of any of the foregoing values, or a range encompassing any of the foregoing values. In one aspect the dose of the cytidine analog is 134 mg/m².

In a further aspect, a disclosed pharmaceutical composition comprises at least one substituted triphenylacetamide analog, e.g., 2,2-bis(4-fluorophenyl)-2-phenylacetamide, in a unit dose that can be administered more than once a day, for example, 2, 3, 4, 5 or 6 times a day. In various aspects, a disclosed pharmaceutical composition comprises at least one substituted triphenylacetamide analog, e.g., 2,2-bis(4-fluorophenyl)-2-phenylacetamide, in a unit dose that can be administered 1 or 2 times per day, so that the total dosage for a 70 kg adult is in the range of 0.001 to about 15 mg per kg weight of subject per administration. In a further aspect, a disclosed pharmaceutical composition comprises at least one substituted triphenylacetamide analog, e.g., 2,2-bis(4-fluorophenyl)-2-phenylacetamide, in a unit dose that is 0.01 to about 1.5 mg per kg weight of subject per administration, and such therapy can extend for a number of weeks or months, and in some cases, years. It will be understood, however, that the specific dose level for any particular patient will depend on a variety of factors including the activity of the specific compound employed; the age, body weight, general health, sex and diet of the individual being treated; the time and route of administration; the rate of excretion; other drugs that have previously been administered; and the severity of the particular disease undergoing therapy, as is well understood by those of skill in the area.

In a further aspect, the disclosed pharmaceutical composition has a unit dose form comprising at least one substituted triphenylacetamide analog, e.g., 2,2-bis(4-fluorophenyl)-2-phenylacetamide, that when administered to a subject provides an area-under-the-curve value for the at least one substituted triphenylacetamide analog, e.g., 2,2-bis(4-fluorophenyl)-2-phenylacetamide, of from about 100 ng·hr/mL to about 400 ng·hr/mL, or of about 100, 150, 200, 225, 250, 275, 300, 325, 350, 375, or about 400 ng·hr/mL, or a combination of any of the foregoing values, or a range encompassing any of the foregoing values. In a still further aspect, the foregoing pharmaceutical composition is formulated for oral administration.

In a further aspect, the disclosed pharmaceutical composition has a unit dose form comprising at least one substituted triphenylacetamide analog, e.g., 2,2-bis(4-fluorophenyl)-2-phenylacetamide, that when administered to a subject a maximum plasma concentration of the at least one substituted triphenylacetamide analog, e.g., 2,2-bis(4-fluorophenyl)-2-phenylacetamide, from about 0.005 to about 0.05 μM following administration to the subject, or of about 0.005, 0.005, 0.01, 0.025, or about 0.05 μM, or a combination of any of the foregoing values, or a range encompassing any of the foregoing values. In another aspect, the disclosed pharmaceutical composition has a unit dose form comprising at least one substituted triphenylacetamide analog, e.g., 2,2-bis(4-fluorophenyl)-2-phenylacetamide, that when administered to a subject achieves a maximum plasma concentration at from about 60 minutes to about 180 minutes following administration to a subject, or at about 60, 75, 90, 105, 120, 135, 150, 165, or about 180 minutes following administration to a subject, or a combination of any of the foregoing values, or a range encompassing any of the foregoing values. In a still further aspect, the foregoing pharmaceutical composition is formulated for oral administration.

In one aspect, the disclosed pharmaceutical compositions can be co-administered with a disclosed second therapeutic agent that is used with the at least one substituted triphenylacetamide analog, e.g., 2,2-bis(4-fluorophenyl)-2-phenylacetamide, and a second therapeutic agent, such as, for example, a hydroxyurea analog; a DNMT1 inhibitor; a gene therapy agent; an HBS polymerization inhibitor, including, but not limited to, voxelotor (previously designated as GBT440 during pre-clinical and clinical research); an erythroid maturation agent, including, but not limited to, luspatercept; a therapeutic agent that binds P-selectin (“a P-selectin binder”) such as monoclonal antibody such as crizanlizumab; a pyruvate kinase M2 activator, an iron chelator, and/or an HDAC inhibitor. As used herein, it is understood that a second co-administered therapeutic agent can be co-formulated in the same dosage form, or co-administered as a separate dosage form or by a non-oral route of administration, e.g., by intravenous infusion or subcutaneous injection or the like, it can be administered in a dosage of from about 10 to about 500 mg/m² of body surface area, or at about 10, 25, 50, 75, 100, 150, 200, 250, 300, 350, 400, 450, or about 500 mg/m² of body surface area, or a combination of any of the foregoing values, or a range encompassing any of the foregoing values. It is further understood that co-administered can be simultaneous co-administration or sequential co-administration of the at least one substituted triphenylacetamide analog, e.g., 2,2-bis(4-fluorophenyl)-2-phenylacetamide, and the second therapeutic agent.

In a further aspect, the disclosed pharmaceutical compositions comprise a disclosed the second therapeutic agent that is administered in tablet or capsule form, the second therapeutic agent is administered in dosage of from about 1 to about 400 mg/kg of body weight, or at about 1, 5, 10, 15, 20, 25, 50, 75, 100, 125, 150, 175, 200, 225, 250, 275, 300, 325, 350, 375, or about 400 mg/kg of body weight, or a combination of any of the foregoing values, or a range encompassing any of the foregoing values.

It can be necessary to use dosages outside these ranges in some cases as will be apparent to those skilled in the art. Further, it is noted that the clinician or treating physician will know how and when to start, interrupt, adjust, or terminate therapy in conjunction with individual patient response.

### Methods for Treating a Disorder

In various aspects, disclosed herein are methods of treating a disorder by administering a disclosed pharmaceutical composition. In a further aspect, the disorder treated is a liver disease (e.g., nonalcoholic fatty liver disease or “NASH”), fibrosis (e.g., renal fibrosis), a hematological disorder, a neurological disorder, a viral infection, or a cancer, by administering the disclosed pharmaceutical compositions to a subject. Examples of a hematological disorder, as used herein, include a sickling blood disorder, a thalassemia, or an anemia, e.g., hereditary stomacytosis; and examples of a neurological disorder, as used herein, include Alzheimer's disease and Parkinson's disease.

In a further aspect, disclosed herein are methods of treating a hematological disorder associated with a sickling blood disorder, e.g., sickle cell disease, and/or a thalassemia in a subject, the method comprising administering a therapeutically effective amount of a disclosed pharmaceutical composition or a disclosed therapeutic agent to a subject. In a further aspect, the method comprises administering a therapeutically effective amount of a disclosed pharmaceutical composition comprising at least one substituted triphenylacetamide analog, e.g., 2,2-bis(4-fluorophenyl)-2-phenylacetamide. In a still further aspect, the method comprises administering a disclosed pharmaceutical composition comprising a therapeutically effective amount of comprising at least one substituted triphenylacetamide analog, e.g., 2,2-bis(4-fluorophenyl)-2-phenylacetamide, and a therapeutically effective amount of a second therapeutic agent, e.g., a hydroxyurea analog; a DNMT1 inhibitor; a gene therapy agent; an HBS polymerization inhibitor, including, but not limited to, voxelotor (previously designated as GBT440 during pre-clinical and clinical research); an erythroid maturation agent, including, but not limited to, luspatercept; a therapeutic agent that binds P-selectin (“a P-selectin binder”) such as monoclonal antibody such as crizanlizumab; a pyruvate kinase M2 activator, an iron chelator, and/or an HDAC inhibitor. In a yet further aspect, the method comprises administering a therapeutically effective amount of at least one substituted triphenylacetamide analog, e.g., 2,2-bis(4-fluorophenyl)-2-phenylacetamide, formulated for oral administration.

In a further aspect, the method for treating a hematological disorder further comprises the step of identifying a subject having a hematological disorder. In a yet further aspect, the subject in the method for treating a hematological disorder has already been identified as having a hematological disorder. In a still further aspect, the hematological disorder can be sickle cell disease or thalassemia.

In a further aspect, the method for treating a hematological disorder comprises administering a therapeutically effective amount of at least one substituted triphenylacetamide analog, e.g., 2,2-bis(4-fluorophenyl)-2-phenylacetamide, of from about 0.1 to about 150 mg/m², or can be from about 10 to about 150 mg/m², or can be about 0.1, 0.5, 1, 5, 10, 15, 20, 25, 30, 35, 40, 45, 50, 55, 60, 65, 70, 76, 80, 85, 90, 95, 100, 105, 110, 115, 120, 125, 130, 135, 140, 145, or about 150 mg/m², or a combination of any of the foregoing values, or a range encompassing any of the foregoing values.

From the foregoing, it will be seen that aspects herein are well adapted to attain all the ends and objects hereinabove set forth together with other advantages which are obvious and which are inherent to the structure.

While specific elements and steps are discussed in connection to one another, it is understood that any element and/or steps provided herein is contemplated as being combinable with any other elements and/or steps regardless of explicit provision of the same while still being within the scope provided herein.

It will be understood that certain features and subcombinations are of utility and may be employed without reference to other features and subcombinations. This is contemplated by and is within the scope of the claims.

Since many possible aspects may be made without departing from the scope thereof, it is to be understood that all matter herein set forth or shown in the accompanying drawings and detailed description is to be interpreted as illustrative and not in a limiting sense.

It is also to be understood that the terminology used herein is for the purpose of describing particular aspects only, and is not intended to be limiting. The skilled artisan will recognize many variants and adaptations of the aspects described herein. These variants and adaptations are intended to be included in the teachings of this disclosure and to be encompassed by the claims herein.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### Examples

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in or is at ambient temperature, and pressure is at or near atmospheric.

1. Screening of Formulation Components Based on Saturation Solubility Studies.

An excess amount of senicapoc is added to 1 g of each vehicle, followed by mixing (100 rpm) in a shaking incubator (Infors AG, Bottmingen, Switzerland) at 37° C. for 48 h. Afterward, the equilibrated samples are centrifuged (Eppendorf centrifuge 5804 R, Hamburg, Germany) at 4000×g for 30 min (37° C.) to remove any insoluble senicapoc. The concentration of senicapoc in supernatants is measured by HPLC-UV (Shimadzu C 204353, Kyoto, Japan) after dilution with acetonitrile.

2. Screening of Surfactants and Cosurfactants for Emulsifying Ability.

The emulsification ability of various surfactants can be screened as described by Date and Date and Nagarsenker (2007) with minor modifications. In brief, selected oils and surfactants are mixed 1.1 (w/w), heated at 40-45° C., and are then homogenized as described above. The mixture (500 mg) is accurately weighed and dispersed into 10 mL of deionized water under gentle stirring. Visual evaluation is used to assess the relative turbidity. The resulting dispersions are allowed to stand for 2 h, and their transmittance values are measured at a wavelength of 550 nm using a NanoDrop™ 2000 spectrophotometer (Thermo Fisher Scientific) against deionized water as a control. Various cosurfactants are screened by mixing the surfactant with each selected cosurfactant in a 2:1 (w/w) ratio. The oily phase is added to this mixture in a 1:3 ratio and homogenized with the aid of gentle stirring and heat (40-45° C.), The resulting dispersions are accessed for different parameters as mentioned for the surfactant screening.

3. Preparation of Disclosed Pharmaceutical Compositions Comprising Senicapoc and Drug Content Determination Thereof.

Blank disclosed pharmaceutical compositions, i.e., a formulation that is complete except having omitted active agent, senicapoc, are prepared by mixing the appropriate quantities of oil, surfactant and cosurfactant under agitation (100 rpm, 35 min). Then, 10 mg of senicapoc is added to 600 mg of each blank disclosed pharmaceutical composition and mixed under agitation (100 rpm, 35 min) for dissolution until a transparent preparation is obtained. To determine the maximum loading content of senicapoc in each formulation, an excess amount of senicapoc is added to 1 g of each blank disclosed pharmaceutical composition by mixing (100 rpm) in a shaking incubator at 37° C. for 48 h. The equilibrated samples are centrifuged at 4000 g for 30 min to remove the excess senicapoc, and the concentration of senicapoc in the supernatant is determined by HPLCUV after appropriate dilution with acetonitrile.

4, Characterization of Optimized Formulation.

Transmittance Percentage. The percentage transmittance is evaluated as described by Shakeel et al (2013). Briefly, a disclosed pharmaceutical composition (1 g) is nanoemulsified in 100 mL of deionized water and allowed to stabilize for an hour. The transmittance percentage of samples is measured at 550 nm wavelength using a Nanodrop UV spectrophotometer (Thermo Fisher Scientific Inc., Waltham, Mass., USA) against deionized water as a control. It is anticipated that the disclosed pharmaceutical compositions will exhibit a high transmittance values (≥95%) consistent with the clarity of the disclosed pharmaceutical compositions.

Viscosity Measurement. Viscosities of formulations are determined with the aid of a modular compact rheometer (MCR 102, Anton Paar Instruments Ltd, Graz, Australia) equipped with a temperature control system. A parallel plate (50 mm) is used for the measurements. The gap size is set at 500 μm, and 4 μL of each preconcentrated disclosed pharmaceutical composition is used. The shear stress is measured at varying rates from 0.1 to 100 s⁻¹ for 5 min. All rheological measurements are made at 25° C., and data are analyzed with Rheocompass software (version 1.13.44-release, Anton Pear Instruments Ltd, Graz, Australia).

Emulsification Time. One gram of each disclosed pharmaceutical composition is added to 500 mL of 0.1 HCl and maintained at 37±0.5° C. under gentle agitation (100 rpm). The time required in seconds to obtain a clear dispersion is recorded as the emulsification time (Basalious et al., 2010).

In the characterization of the disclosed pharmaceutical compositions using the foregoing exemplary method, it is anticipated that these formulations will have an emulsification time to be less than 100 s. This result is consistent with the disclosed pharmaceutical compositions having an ability to disperse completely and quickly when subjected to aqueous dilution under mild agitation.

5. Determination of Size and Zeta Potential.

Average Globule Size and Polydispersity Index (PDI). The average globule size and polydispersity index (PDI) of the disclosed pharmaceutical compositions can be determined by dynamic light scattering (DLS) at 37° C. using a Nano ZS system (Malvern Instruments Ltd, UK). To prepare samples, 600 mg of a disclosed pharmaceutical composition is dispersed in 200 mL of deionized water, hydrochloric acid buffer pH 1.2, PBS pH 6.8, FaSSGF pH 1.6 and FaSSIF pH 6.8. The droplet size and PDI of the resulting emulsions are directly measured. Zeta potential is measured by electrophorefic mobility (PCS) using a Zetasizer Nano ZS system (Malvern Instruments Ltd, UK). After diluting the SNEDDS formulation (600 mg) with 200 mL of deionized water, the samples are directly measured.

Thermodynamic Stability Studies. The formulations are subjected to heating-cooling cycles. Six cycles between 4° C. and 40° C. are applied with storage at each temperature for not less than 48 h. Those formulations, which are stable (no phase separation) are subjected to freeze-thaw cycles involved three cycles between −21° and 20° C. with storage at each temperature for not less than 48 h. Further, centrifugation is performed at 4000 g for 30 min to observe phase separation.

It is anticipated that the disclosed pharmaceutical compositions will demonstrate using the foregoing method, high stability and limited phase separation.

6. In Vitro Dissolution Profile.

Dissolution studies are performed using a drug dissolution tester (Sotax AT7, CH-4008 Basel, Switzerland) according to US Apparatus II (paddle method). Pure senicapoc and senicapoc-SNEDDS formulations (6 lt00 mg) equivalent to 10 mg filled in size “0” hard gelatin capsules (Capsugel Inc., Morristown, N.J., USA) are placed in 500 mL of USP buffer (pH 1.2) used as dissolution media. A paddle rotation speed of 100 rpm and a temperature of 37±0.5° C. are used. At predefined time intervals (5, 10, 20, 30, 40, 50 and 60 min), a 2 mL aliquot is withdrawn and replenished with a similar volume of fresh blank media. The withdrawal samples are filtered through 0.22 μm Rotilabo® syringe filters (Carl Roth, Karlsruhe, Germany) and transferred into glass vials. Then, 10 μL of the resulting filtrate is quantified by HPLC-UV to measure the concentration of senicapoc.

It is anticipated that the disclosed pharmaceutical compositions will be associated with increased senicapoc solubility and release of senicapoc in the foregoing test system,

7. In Vitro Lipolysis.

Lipolysis experiments are carried out according to the procedure described by Crum et al. (2016) with minor adjustments. The experimental setup consisted of a T5 Mettler Toledo pH-stat titration unit (Greifensee, Switzerland) comprising a combined pH Ag/AgCl electrode (DGI 115-SC) and coupled to a 30 mL DV 1020 Mettler Toledo auto-burette (Greifensee, Switzerland), an IKA C-MAG HS7 thermostat-jacketed glass reaction vessel (Staufen, Germany) and a compact stirrer (Mettler Toledo).

The disclosed pharmaceutical compositions are gently dispersed into 40 mL of digestion buffer (comprising 1.4 mM CaCl2·2H2O, 2 mM Tris-maleate, 150 mM NaCl, 3 mM NaTDC, and 0.75 mM TLC). After 15 min, the pH is automatically adjusted to 6.5±0.05 with 0.5 M NaOH. The in vitro lipolysis is initiated by the addition of 4 mL of pancreatin extract containing lipase (lipase activity equivalent to 8×USP specifications) and other pancreatic enzymes (amylase, protease and ribonuclease). The pancreatin extract is freshly prepared before each experiment by mixing 1 g of pancreatic powder with 5 mL of digestion buffer and 20 μL of 0.5 M NaOH solution to reach the target pH 6.5. The resulting enzyme suspension is centrifuged (4000 g, 4° C., Eppendorf centrifuge 5804 R, Hamburg, Germany) for 15 min.

During the experiment, the released fatty acids are automatically titrated with 0.5 M NaOH to maintain pH 6.5. Two milliliters of digestion medium is withdrawn in 5 min intervals up to 60 min. The lipase activity is inhibited by the addition of 10 μL of 1.0 M 4-bra-mophenylboronic acid (in methanol). The samples are vortexed and centrifuged (6700 g, 4 MiniSpin, Eppendorf AG, Hamburg, Germany) for 15 min, resulting in the separation of the digestion content in a clear supernatant and off-white pellet. The drug content in the supernatant is quantified by HPLC-UV following appropriate dilution with acetonitrile. Lipolysis is also performed with blank digestion medium in the absence of an added disclosed pharmaceutical composition.

It is anticipated that using the foregoing method for in vitro lipolysis determination that the disclosed pharmaceutical compositions will demonstrate an elevated level of senicapoc solubilization in the aqueous phase after 60 min of in vitro lipolysis and reduced drug precipitation during digestion.

It will be apparent to those skilled in the art that various modifications and variations can be made in the present disclosure without departing from the scope or spirit of the disclosure. Other embodiments of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

1. A pharmaceutical composition comprising a therapeutically effective amount of a compound of a first therapeutic agent, and at least one pharmaceutically acceptable carrier;
wherein the first therapeutic agent comprises at least one substituted triphenyl acetamide analog having a structure represented by a formula: 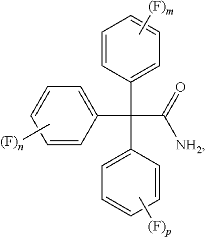
wherein m, n and p can be independently selected from 0 and 1 and at least one of m, n and p can be 1, or a pharmaceutically acceptable salt, solvate, or polymorph thereof;
wherein the pharmaceutically acceptable carrier comprises at least one oil or lipidic material, at least one surfactant, and at least one hydrophilic co-surfactant; and
wherein the least one oil or lipidic material, the least one surfactant, and the least one hydrophilic co-surfactant form a suspension, an emulsion, or a microemulsion.

2. The pharmaceutical composition of claim 1, wherein the first therapeutic agent has a structure represented by a formula:

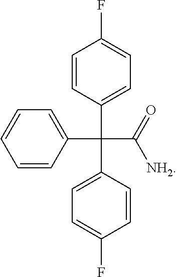 [SMILES: NC(=O)C(c1ccccc1)(c1ccc(F)cc1)c1ccc(F)cc1]

3. The pharmaceutical composition of claim 1, further comprising a second therapeutic agent selected from pentoxifylline, pentosan polysulfate sodium, voxelotor, 5-hydroxymethyl-2-furfural (5-HMF), a hydroxyurea analog; a DNMT1 inhibitor; a Janus kinase (JAK) inhibitor; a gene therapy therapeutic agent; an HbS polymerization inhibitor; an erythroid maturation agent; an fetal hemoglobin induction agent; a P-selectin binding agent; a pyruvate kinase M2 activator; a PDE9 inhibitor; an activator or agonist of soluble guanylate cylcease; an iron chelator; an anti-hepcidin therapeutic agent; an HDAC inhibitor; and combinations thereof.

4.-12. (canceled)

13. The pharmaceutical composition of claim 1, further comprising a surfactant or co-surfactant selected from Cremophor-EL® (polyoxyl-35 castor oil), polyethylene glycol 400 (PEG 400), Labrafil M® 1944 CS (oleoyl polyoxyl-6-glycerides), Labrafil M® 2125 CS (linoleoyl polyoxyl-6-glycerides), Labrasol AFL® (caprylocaproyl polyoxyl-8-glycerides), Transcutol HP® (diethylene glycol monoethyl), Capryol®90 (propylene glycol monocaprylate type II), Capryol PGMC® (propylene glycol monocaprylate type 1), Labrafac lipophile WL® 1349 (triglycerides medium chain), Lauroglycol®90 (propylene glycol monolaurate) and Maisine® 35-1 (glycerol monolinoleate), Tween® 80 (polysorbate 80), Tween®20 (polysorbate 20), sodium taurodeoxycholate (NaTDC), L-α-phosphatidylcholine (TLC), 4bromophenylboronic acid, Triton X-100, Propylene glycol and oleic acid.

14. The pharmaceutical composition of claim 1, wherein the pharmaceutical composition is an enteric coated and stable gastric or stomach pH.

15. The pharmaceutical composition of claim 1, wherein the first therapeutic agent is present in an amount of from about 1 mg to about 50 mg in a single dosage form.

16. The pharmaceutical composition of claim 1, wherein the pharmaceutical composition is formulated as an orally administered dosage form.

17. The pharmaceutical composition of claim 16, wherein the orally administered dosage form comprises a layered tablet, a tablet-in-tablet form, a tablet-in-capsule form, or a capsule-in-capsule form, granule, powder in sachet or bag, capsule, tablet, pill, or other oral solid dosage form.

18. The pharmaceutical composition of claim 17, wherein the orally administered dosage form is a capsule.

19. The pharmaceutical composition of claim 18, wherein the at least one substituted triphenyl acetamide analog is present in an amount of about 2 mg to 10 mg in the orally administered dosage form.

20. The pharmaceutical composition of claim 18, wherein the at least one substituted triphenyl acetamide analog is present in an amount of about 5.5% to about 15% based on the total weight of the composition.

21.-24. (canceled)

25. The pharmaceutical composition of claim 1, wherein the pharmaceutical composition has a semi-solid physical state at about 15° C. to about 30° C.; and wherein the pharmaceutical composition has a physical state at about 36° C. or greater that is a gel, paste, and/or liquid.

26. The pharmaceutical composition of claim 1, wherein the pharmaceutical composition has a droplet size of from about 20 nm to about 200 nm.

27.-30. (canceled)

31. A method for treating a hematological disorder, cancer, neurological disorder in a subject, the method comprising administering an effective amount of the pharmaceutical composition of claim 1 to the subject.

32. The method of claim 31, wherein the hematological disorder comprises sickle cell disease, thalassemia, anemia, or blood cancer.

33. The method of claim 31, wherein the neurological disorder comprises Alzheimer's Disease or brain inflammation.

34. (canceled)

35. (canceled)

36. A method for treatment of sickle cell disease, thalassemia, anemia or increase total hemoglobin in a subject comprising:
administering to the subject a selective Gardos channel blocker;
wherein the selective Gardos channel blocker selectively inhibits the efflux of potassium from the erythrocytes.

37. The method of claim 36, wherein the Gardos channel blocker is selected from imidazole antimycotics, clotrimazole, metronidazole, econazole, arginine, Tram-34, harybdotoxin, nifedipine, 2,2-Bis(4-fluorophenyl)-N-methoxy-2-phenylacetamidine, 2-(2-Chlorophenyl)-2,2-diphenylacetaldehyde oxime, 2-(2-Chlorophenyl)-2,2-bis(4-fluorophenyl)-N-hydroxyacetamidine, 2,2,2-Tris(4-fluorophenyl)-N-hydroxyacetamidine, 2-(2-Fluorophenyl)-2-(4-fluorophenyl)-N-hydroxy-2-phenylacetamidine, phosphoric acid 3-(2-oxazolyl)-4-[3-(trifluoromethyl)phenylsulfonamido]phenyl monoester, N-[2-(4,5-Dihydrooxazol-2-yl)phenyl]-3-(trifluoromethyl)benzenesulfonamide, N-[4-Methoxy-2-(2-oxazolyl)phenyl]benzenesulfonamide, N-[4,5-Dimethoxy-2-(3-methyl-I,2,4-oxadiazol-5-yl)phenyl]-3-(trifluoromethyl)benzenesulfonamide, N-[2-(2-Furyl)phenyl]-3-(trifluoromethyl)benzenesulfonamide, N-[4-Methyl-2-(2-oxazolyl)phenyl]-3-(trifluoromethyl)benzenesulfonamide), 2,2-bis(4-fluorophenyl)-2-phenylacetamide, and combinations thereof.

38.-42. (canceled)

43. A composition comprising a self-microemulsifying excipient formulation for increasing the bioavailability of substituted triphenyl acetamide analog having a structure represented by a formula: 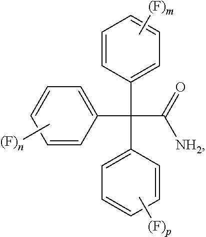
wherein m, n and p can be independently selected from 0 and 1 and at least one of m, n and p can be 1, or a pharmaceutically acceptable salt, solvate, or polymorph thereof;
the self-microemulsifying excipient formulation comprising an emulsion including an oil or other lipid material, a surfactant, and a hydrophilic co-surfactant, said hydrophilic co-surfactant having a hydrophilic-lipophilic balance (HLB) greater than 8.

44. The composition of claim 43, wherein the hydrophilic co-surfactant has a hydrophilic-lipophilic balance (HLB) between approximately 10 and 15.

45.-46. (canceled)
